(19) United States
(12) Patent Application Publication
(10) Pub. No.: US 2015/0201846 A1
(43) Pub. Date: Jul. 23, 2015

(54) DEVICE AND METHOD FOR PROVIDING INFORMATION INDICATIVE OF A STRESS SITUATION IN A HUMAN

(71) Applicants: DAVID MAIERSHON, Hod Hasharon (IL); ORI AKSTEIN, Hod Hasharon (IL)

(72) Inventors: David Maiershon, Hod Hasharon (IL); Ori Akstein, Hod Hasharon (IL)

(73) Assignee: Medbright Medical Solutions Ltd, Hod Hasharon (IL)

(21) Appl. No.: 14/415,806

(22) PCT Filed: Jul. 22, 2013
(86) PCT No.: PCT/IB2013/056013

§ 371 Date: Jan. 20, 2015

(30) Foreign Application Priority Data
Jul. 24, 2012 (GB) 1213159.5

(51) Int. Cl. A61B 5/0205; A61B 5/00; A61B 5/1455; G01N 33/49; G06F 19/00

(52) U.S. Cl. CPC A61B 5/0205; G01N 33/4925; G06F 19/3418; A61B 5/14551; A61B 5/7285; A61B 5/746; A61B 5/742; A61B 5/0022; A61B 5/024

(57) ABSTRACT

Disclosed are methods and devices for monitoring and sensing stress situations of a human, such as a baby, and in some embodiments informing parents or other caretakers of such situations.

### RELATED APPLICATION

The present application gains priority from UK Patent Application GB 1213159.5 filed 24 Jul. 2012, which is incorporated by reference as if fully set-forth herein.

### FIELD AND BACKGROUND OF THE INVENTION

The invention, in some embodiments, relates to the field of sensors, and more particularly to methods and devices for providing information indicative of a stress situation in a baby, and in some embodiment, notifying a caregiver of such a situation.

Electro-optical measurement of blood characteristics has been found to be useful in many areas of blood constituent diagnostics, such as glucose levels, oxygen saturation, hematocrit, billirubin and others. This method is advantageous in that it can be performed in a non-invasive fashion. In particular, much research has been done on oximetry, a way of measuring oxygen saturation in the blood, as an early indicator of respiratory distress.

Pulse oximetry uses the difference in the absorption properties of oxyhemoglobin and deoxyhemoglobin to measure blood oxygen saturation in arterial blood. The oximeter passes light, usually red and infrared, through body tissue and uses a photodetector to sense the absorption of light by the tissue. By measuring oxygen levels in the blood, one is able to detect respiratory distress at its onset.

Various types of pulse oximetry are known. In transmission oximetry, two or more wavelengths of light are transmitted by one or more light sources on one side of an appendage where blood perfuses the tissue thereof (i.e. a finger or earlobe) through the appendage towards a photodetector on the opposite side of the appendage, allowing to determine how much of what wavelengths of light are absorbed by blood in the appendage. The light source(s) and photodetector are typically mounted on a clip or on a band that attaches to the appendage and delivers data by cable to a processor. There are many problems with this type of pulse oximetry, including discomfort caused by the clips or bands, geometrical limitations of the appendage to which a clip or band can be attached, and susceptibility to inaccurate reading due to movement of the light source(s) with respect to the photodetector.

In reflective, or backscattering, pulse oximetry, the light sources and photodetector are placed side by side on the same tissue surface, so reflective pulse oximeters may also be placed on a surface of a head, wrist or foot.

Electrodermal activity (EDA) sensors detect changes in the electrical properties of the skin in response to stress or anxiety. EDA sensors measure electrical properties of the skin by recording the electrical resistance of the skin following passing a low voltage current or by recording weak currents generated by the body. The electrodermal activity of a human may change in response to emotions and emotional states such as fear, anger, startle response, pain, and orienting response, which allows EDA sensing to be used in lie detection tests such as the polygraph.

During the first year of life, human infants are susceptible to breathing disturbances and respiratory distress. Sudden Infant Death Syndrome (SIDS), also known as crib death or cot death, is a medical condition in which an infant enters respiratory distress and stops breathing, leading to the death of the infant. Although the cause and the warning signs of SIDS are not clear, it has been shown that early detection of respiratory distress can provide the time to administer the aid necessary to prevent death.

Many types of baby-monitoring devices are available, from devices including simple motion detectors to complicated devices which stream oxygen enriched air into the infant's environment. Some of the more accepted baby-monitoring devices include chest motion monitors, carbon dioxide level monitors and heart rate (pulse) monitors. Unfortunately such devices often do not give the advance warning necessary for a caregiver to administer aid. In addition, such baby-monitoring devices often require attaching straps and/or cords to the baby, which are cumbersome to use and present a strangulation risk.

The most commonly used baby-monitoring device is a chest motion monitor configured to issue a warning when no chest motion is detected during a predetermined period (e.g., 1 minute) but gives no warning when the breathing patterns become irregular or when hyperventilation is occurring. Such a device typically detects respiratory distress only once chest motion has ceased, when it is usually too late to help the baby. In addition, chest-motion monitors have a high level of “false alarms” due to difficulty in distinguishing between normal lapses in breathing (up to 20 seconds in infants) and respiratory distress.

Many infants, and particularly neonates, sleep for a large portion of the day, often as much of 20 hours. A baby-monitoring device must be battery-powered and comfortable to wear to be available for monitoring a baby during all sleep times and to be cable-free. These requirements mean that a baby-monitoring device is preferably designed to operate in a manner to ensure that the batteries have long operation durations during the monitoring time.

### SUMMARY OF THE INVENTION

The invention, in some embodiments, relates to the field of sensors, and more particularly to methods and devices for providing information indicative of a stress situation in a human, such as a human baby.

The invention, in some embodiments, relates to the field of sensors, and more particularly to methods and devices for identifying a possible reason for a potential stress situation, and in some embodiments notifying a caregiver such as a parent of the identified reason.

According to an aspect of some embodiments of the invention, there is provided a method for providing information indicative of a stress situation in a human, comprising:
positioning a pulse oximeter and an electrodermal activity (EDA) sensor to monitor a human;
at a processor functionally associated with the EDA sensor and the pulse oximeter, monitoring the human by receiving from the EDA sensor an EDA measurement signal at a EDA sensing rate;
at a processing rate, processing the received EDA measurement signals to identify an EDA indication of stress; and
if an EDA indication of stress is identified:
   activating the pulse oximeter to determine a measure of at least one of pulse and blood oxygenation of the human;
   at the processor, receiving from the pulse oximeter at least one pulse oximeter signal related to the determined measure of at least one of pulse and blood oxygenation;
   at the processor, processing the received pulse oximeter signal to identify a pulse oximeter indication of stress; and
   if a pulse oximeter indication of stress is identified, the processor automatically providing an alarm signal.

In some embodiments of the method, the providing an alarm signal comprises at least one of: providing a visual alarm from a visual indicator functionally associated with the processor; providing an audible alarm from an aural indicator functionally associated with the processor; and automatically activating a wireless transmitter functionally associated with the processor to wirelessly transmit an alarm signal to a remote monitoring unit.

In some embodiments, the method also comprises at the remote monitoring unit, providing an alarm perceivable to a human.

In some embodiments, the remote monitoring unit includes at least one of: a component suitable to be worn by a person; a component configured to be clipped onto clothes of a person; and a component configured to be carried in a pocket of a person.

In some embodiments, the remote monitoring unit comprises a component not configured to be ordinarily worn by a person, and the method also comprising placing the component not configured to be worn by a person in the vicinity of a person.

In some embodiments, the method also comprises, during the monitoring of the human, if no EDA indication of stress is identified, wirelessly transmitting an all-clear signal to the monitoring unit.

In some embodiments, the pulse oximeter operates with power from an energy storage unit, and the method also comprises: during the monitoring of the human, monitoring a charge state of the energy storage unit; and if the charge state of the energy storage unit is below a charge threshold, the processor automatically activating the wireless transmitter to wirelessly transmit a low battery signal to the remote monitoring unit.

In some embodiments, the method also comprises: in the remote monitoring unit, monitoring quality of signals received from the wireless transmitter; and if the quality of the signals is insufficient, providing an insufficient signal indication perceivable to a human.

In some embodiments, the measure of blood oxygenation comprises a blood oxygenation percentage.

In some embodiments, the pulse oximeter indication of stress comprises at least one of: a blood oxygenation measure of the pulse oximeter signal being below a blood oxygenation threshold; a pulse rate measure of the pulse oximeter signal being below a low pulse rate threshold; and a pulse rate measure of the pulse oximeter signal being above a high pulse rate threshold.

In some embodiments, the method also comprises indicating whether the pulse oximeter indication is indicative of stress stemming from a problem in blood oxygenation of the human or of stress stemming from a problem in pulse rate of the human.

In some embodiments, the method also comprises storing the EDA measurement signals and/or the measures of pulse and/or blood oxygenation in a memory.

In some embodiments, the method also comprises, during the monitoring of the human, if no EDA indication of stress is identified, keeping the pulse oximeter in an inactive energy-saving state.

According to an aspect of some embodiments of the invention, there is also provided a device useful for providing information indicative of a situation in a human, comprising:
a pulse-oximeter configured for determining a measure of at least one of pulse and blood oxygenation of a human and producing a pulse oximeter signal related to the determined measure of at least one of pulse and blood oxygenation;
an electrodermal activity (EDA) sensor configured for determining electrodermal activity of a human at an EDA sensing rate and producing an EDA measurement signal related to the determined electrodermal activity;
a processor functionally associated with the pulse oximeter and with the EDA sensor; and
a wireless transmitter functionally associated with the processor,
wherein the processor is configured to:
   receive from the EDA sensor an EDA measurement signal at the EDA sensing rate,
   at a processing rate, process the received EDA measurement signals to identify an EDA indication of stress,
   if an EDA indication of stress is identified, activate the pulse oximeter to determine a measure of at least one of pulse and blood oxygenation of a human,
   receive from the pulse oximeter at least one pulse oximeter signal related to the determined measure of at least one of pulse and blood oxygenation,
   process the received the pulse oximeter signal to identify a pulse oximeter indication of stress, and
   if a pulse oximeter indication of stress is identified, automatically provide an alarm signal.

In some embodiments, the device also comprises at least one indicator configured, upon provision of the alarm signal, to provide at least one indication perceivable to a human. In some such embodiments, at least one indicator is a visual indicator to provide a visual indication to the human. In some such embodiments, at least one indicator is an aural indicator to provide an audible indication to the human.

In some embodiments, the device also comprises a remote monitoring station, including:
a wireless receiver configured to receive transmissions from the wireless transmitter; and
an alarm indicator, configured to provide an alarm perceivable to a human upon receipt of an alarm signal by the wireless receiver.

In some embodiments, the human is a baby.

In some embodiments, the remote monitoring unit comprises at least one of: a component suitable to be worn by a person; a component configured to be clipped onto clothes of a person; a component configured to be carried the pocket of a person; and a component not configured to be ordinarily worn by a person.

In some embodiments, the remote monitoring unit of the device comprises at least one of:

a visual indicator, wherein the alarm perceivable to a human comprises a visual alarm;

an aural indicator, wherein the alarm perceivable to a human comprises an audible alarm; and

a tactile indicator and the alarm perceivable to a human comprises a tactile alarm.

In some embodiments, the processor of the device is also configured to monitor a charge state of an energy storage unit of the device, and if the charge state of the energy storage unit is below a charge threshold, to transmit a low battery signal, via the transmitter of the device.

In some embodiments, the processor of the device is also configured, if no EDA indication of stress is identified, to wirelessly transmit an all-clear signal, via the transmitter.

In some embodiments, the remote monitoring unit of the device is configured to monitor quality of signals received from the wireless transmitter and to provide an insufficient signal indication perceivable to a human if the quality of signals is insufficient.

In some embodiments of the device, the pulse oximeter indication of stress comprises at least one of:

a blood oxygenation measure of the pulse oximeter signal being below a blood oxygenation threshold;

a pulse rate measure of the pulse oximeter signal being below a low pulse rate threshold; and

a pulse rate measure of the pulse oximeter signal being above a high pulse rate threshold.

In some embodiments of the device, the alarm signal indicates whether the pulse oximeter indication of stress is indicative of stress stemming from a problem in blood oxygenation of the human or of stress stemming from a problem in pulse rate of the human.

In some embodiments, the device also comprises a memory functionally associated with the processor, configured for storing the EDA measurement signals and/or the measures of pulse and/or blood oxygenation.

In some embodiments, the device also comprises an energy storage unit configured to power a group of components including at least one of the EDA sensor, the pulse oximeter, the processor, and the wireless transmitter.

In some embodiments, the energy storage unit is configured to provide sufficient power for continuous operation of the group of components for a period of time of not less than 24 hours of normal use. In some such embodiments, such an energy storage unit has a capacity of at least 130 ampere hours.

In some embodiments, the device is configured to keep the pulse oximeter in an inactive energy-saving state as long as no EDA indication of stress is identified.

In some embodiments, providing an alarm signal comprises providing a visual alarm indication perceivable to a human from a visual indicator functionally associated with the processor. In some embodiments, providing an alarm signal comprises providing an audible alarm signal from an aural indicator functionally associated with the processor.

In some embodiments, providing an alarm signal comprises automatically activating a wireless transmitter functionally associated with the processor to wirelessly transmit an alarm signal to a remote monitoring unit, and in the remote monitoring unit, providing an alarm perceivable to a human. In some such embodiments, the alarm at the remote monitoring unit comprises at least one of a visual alarm, an audible alarm and a tactile alarm.

The EDA sensor and the pulse oximeter may positioned at any suitable location on the human. That said, in some embodiments, the EDA sensor is positioned to be touching the skin of the human. In some embodiments, the pulse oximeter is positioned to be touching the skin of the human.

The sensing rate may be any suitable EDA sensing rate. That said, in some embodiments, the EDA sensing rate is more frequently than every minute. In some embodiments the EDA sensing rate is more frequently than every 30 seconds, 10 seconds, 5 seconds, or even more frequently than every 1 second. In some embodiments, each EDA measurement has a set duration. In some embodiments, the duration is 15 seconds. In some embodiments, the duration is 30 seconds, 1 minute, or even up to five minutes.

The EDA indication of stress may be any suitable EDA indication. That said, in some embodiments, identifying an EDA indication of stress comprises processing a single EDA measurement signal to identify whether the single EDA measurement signal is above an EDA threshold. In some embodiments, identifying an EDA indication of stress comprises processing multiple EDA measurement signals to identify whether a function of the multiple EDA measurement signals is above an EDA threshold. In some such embodiments, the function is an average of multiple EDA measurement signals. In some such embodiments, the function is a mean of multiple EDA measurement signals.

The EDA threshold may be any suitable threshold. That said, in some embodiments, the EDA threshold is a 37% phasic change over the tonic level. In some embodiments, the EDA threshold is a 40% phasic change, a 45% phasic change, and even greater than a 50% phasic change over the tonic level. In some embodiments, the measurement signal is considered to be over the EDA threshold only if it remains over the EDA threshold for a duration of at least 15 seconds.

In some embodiments, during monitoring of the human, if no EDA indication of stress is identified, the pulse oximeter is kept in an inactive energy-saving state. In some embodiments, the pulse oximeter is activated periodically in order to obtain a measurement and make sure that all is well with the baby being monitored.

In some embodiments, the measure of blood oxygenation comprises a blood oxygenation percentage.

In some embodiments, the pulse oximeter signal comprises a single pulse measurement by the pulse oximeter. In some embodiments, the pulse oximeter signal comprises a single blood oxygenation measurement by the pulse oximeter. In some embodiments, the pulse oximeter signal comprises a pulse measurement and a blood oxygenation measurement by the pulse oximeter, obtained at the same time.

In some embodiments, the pulse oximeter signal comprises a function of multiple measurements by the pulse oximeter. In some such embodiments, the function is an average of the multiple measurements by the pulse oximeter. In some such embodiments, the function is a mean of the multiple measurements by the pulse oximeter. In some embodiments, the pulse oximeter signal comprises multiple pulse oximeter signals, each representing a single measurement of pulse, blood oxygenation, or both, by the pulse oximeter.

The pulse oximeter indication of stress may be any suitable pulse oximeter indication. In some embodiments, the pulse oximeter indication comprises a blood oxygenation portion of the pulse oximeter signal being below a blood oxygenation threshold. In some embodiments, the pulse oximeter indication comprises the blood oxygenation portion of at least two pulse oximeter signals being below the blood oxygenation threshold.

The blood oxygenation threshold can be any suitable threshold indicative of human stress. That said, in some embodiments, the blood oxygenation threshold is 60% saturation. In some embodiments, the blood oxygenation threshold is 70% saturation, 80% saturation, or even below 92% saturation.

In some embodiments, the pulse oximeter indication comprises a pulse rate portion of the pulse oximeter signal being below a low pulse rate threshold. In some embodiments, the low pulse rate threshold is 50 beats per minute. In some embodiments the low pulse rate threshold is 45 beats per minute.

In some embodiments, the pulse oximeter indication comprises a pulse rate portion of the pulse oximeter signal being above a high pulse rate threshold. In some embodiments, the high pulse rate threshold is 140 beats per minute. In some embodiments the high pulse rate threshold is 145, 150, or even 160 beats per minute.

In some embodiments, the method also comprises indicating whether the pulse oximeter indication is indicative of stress stemming from a problem in the blood oxygenation of the human or of stress stemming from a problem in the pulse rate of the human.

The wireless transmitter is configured to use any suitable wireless communication method or combination of methods. In some embodiments, the wireless transmitter is configured to use at least one wireless communication method selected from the group consisting of ultrasonic communication, infrared communication, radio-frequency communication, Wi-Fi, GSM, and Bluetooth®.

Any suitable configuration or device may be used as the remote monitoring unit. That said, in some embodiments, the remote monitoring unit includes a component suitable to be worn by a human, such as the human's parent or caregiver. For example, in some embodiments, the remote monitoring unit comprises a bracelet configured to be worn around a human's wrist. In some embodiments, the remote monitoring unit comprises a component which may be clipped onto a clothes of a person, for example formed in the style of a pager or pedometer. In some embodiments, the remote monitoring unit comprises a pendant to be suspended from a human's neck. In some embodiments, the remote monitoring unit comprises a decorative piece of jewelry to be worn on a human's body, such as a ring or an earring. In some embodiments, the remote monitoring unit comprises a worn element to be worn on a human's body, such as an earpiece, eyeglasses, a wristwatch, or a pocket-watch.

In some embodiments, the remote monitoring unit comprises a component that is configured to be carried in a pocket of a person, for example, such as a component outwardly resembling a writing implement.

In some embodiments, the remote monitoring unit comprises a component not configured to be ordinarily worn by a person, which may be placed near a human. In some such embodiments, the method also comprises placing the component not configured to be worn by a person in the vicinity of a person.

In some embodiments, the remote monitoring unit comprises a known mobile communication device modified to function as a remote monitoring unit, to wirelessly receive alarm signals and to provide an alarm perceivable to a human, for example a mobile communication device selected from the group consisting of a mobile telephone, a smart phone, a PDA, a laptop, and a tablet computer.

In some embodiments, the alarm comprises a visual alarm. In some embodiments, the alarm comprises a tactile alarm. In some embodiments, the alarm comprises an audible alarm.

In some embodiments, the method also includes monitoring at least one additional parameter indicative of the human's condition with at least one suitably positioned additional sensor. In some embodiments, the at least one additional sensor is at least one sensor selected from a group consisting of a temperature sensor, a motion sensor, and a heart rate sensor.

In some embodiments, the at least one additional parameter is selected from a group consisting of body temperature monitored with a temperature sensor, body motion monitored with a motion sensor, and heart rate monitored with a heart rate sensor.

In some embodiments, measurements made by at the at least one additional sensor, and/or measurements made by the EDA sensor and/or measurements made by the pulse oximeter, are stored in a memory. In some embodiments, the memory is functionally associated with the wireless transmitter and the method further comprises wirelessly transmitting measurements stored in the memory to a remote location.

In some embodiments, the memory is functionally associated with a port for connection of an external device and the method further comprises:

connecting an external device to the memory through the port; and

retrieving measurements stored in the memory to the external device.

In some embodiments, in a device suitable for implementing the method of the teachings herein, the memory comprises a removable memory, configured to be removed for retrieval of information contained in the memory. In some embodiments, the removable memory is selected from the group consisting of a USB flash drive and a SD card.

In some embodiments, the method also includes providing an energy storage unit configured to power a group of components including at least one of the EDA sensor, the pulse oximeter, the processor, and the transmitter. In some embodiments, the energy storage unit is configured to power a group of components including all of the EDA sensor, the pulse oximeter, the processor, and the transmitter. In some embodiments, the energy storage unit is configured to provide sufficient power for continuous operation of the group of components for a period of time of not less than 24 hours of normal use. In some embodiments, the energy storage unit is configured to provide sufficient power for continuous operation of the group of components for a period of time of not less than 30 hours, 36 hours, or even 48 hours under normal conditions of use. In some embodiments, the energy storage unit is also configured to power at least one of the additional sensor and the memory. In some embodiments, the energy storage unit comprises at least one battery, such as at least one rechargeable battery. In some embodiments, the energy storage unit has a capacity of at least 130 ampere hours.

In some embodiments, in a device implementing the methods of the teachings herein, at least some of the EDA sensor, pulse oximeter, processor, and transmitter, are housed in an assembly of a sensing unit, which assembly is placed on the body of a human. In some such embodiments, the EDA sensor, processor, and transmitter are housed in a first assembly of the sensing unit, and the pulse oximeter is housed in a second assembly of the sensing unit, separate from the first assembly, such that the first and second assemblies are placed on different portions of the body of the human. In some embodiments, all of the EDA sensor, pulse oximeter, processor, and transmitter are housed in a single assembly of the sensing unit, which is placed on the body of the human.

In some such embodiments, the sensing unit also includes the memory. In some embodiments, the sensing unit also includes the at least one additional sensor. In some embodiments, the sensing unit also includes the energy storage unit.

In some embodiments, the energy storage unit is removed from the sensing unit during recharging. In some embodiments, the energy storage unit is recharged while in the sensing unit, for example using a dedicated port.

In some embodiments, the sensing unit comprises a bracelet configured to be worn around the human's wrist or ankle. In some embodiments, the sensing unit comprises a sock to be worn on the human's foot. In some embodiments, the sensing unit comprises a band to be attached around a portion of the human's body, such as the head or the torso. In some embodiments, a sensing unit comprises a band of fabric to be attached to a piece of clothing worn by the human or to a baby's diaper.

In some embodiments, the sensing unit includes a decorated external surface. In some embodiments, the decorations on the external surface comprise decorations configured to captivate a baby's gaze. In some such embodiments, the decorations are in colors configured to capture a neonate's attention such as black, white, or red. In some such embodiments, the decorations comprise images configured to attract a baby's attention.

In some embodiments, the method also includes during monitoring of the human, if no EDA indication of stress is identified, wirelessly transmitting to the monitoring station, via the transmitter, an all-clear control signal. In some such embodiments, the monitoring unit is configured to provide an all-clear indication perceivable by a human. In some embodiments, the all-clear indication comprises a visual indication. In some embodiments, the all-clear indication comprises a tactile indication. In some embodiments, the all-clear indication comprises an audible indication.

In some such embodiment, the all-clear control signal is transmitted periodically. In some embodiments, the all-clear signal is transmitted at a set rate, such as once an hour. In some embodiments, the all-clear signal is transmitted after a set number of the EDA measurements since transmission of a previous all-clear signal.

In some embodiments, the method also comprises during monitoring of the human, monitoring the charge state of the energy storage unit, and if a charge state of the energy storage unit is below a predetermined threshold, producing a low battery signal, wherein the low battery signal is wirelessly transmitted to thee monitoring unit, via the transmitter.

In some such embodiments, the method also comprises in the remote monitoring unit, upon receipt of the low-battery signal, providing a low-battery indication perceivable by a human. In some embodiments, the low-battery indication comprises a visual indication. In some embodiments, the low-battery indication comprises a tactile indication. In some embodiments, the low-battery indication comprises an audible indication.

In some embodiments, the method also comprises in the monitoring unit, monitoring signal quality of signals received from the transmitter, and if the signal quality is insufficient, in the monitoring unit, producing an insufficient signal indication perceivable by a human.

In some embodiments, the method also includes providing an energy storage unit configured to power the remote monitoring unit, wherein the energy storage unit is configured to provide sufficient power for continuous operation of the remote monitoring unit for a period of time of not less than 24 hours under normal conditions of use. In some embodiments, the energy storage unit is configured to provide sufficient power for continuous operation of the remote monitoring unit for a period of time of not less than 30 hours, 36 hours, or even 48 hours under normal conditions of use.

According to an aspect of some embodiments of the invention, there is also provided a method for providing information indicative of a reason for a stress situation in a human, comprising:
positioning an electrodermal activity (EDA) sensor to monitor a human;
at a processor functionally associated with the EDA sensor, receiving from the EDA sensor an EDA measurement signal at a EDA sensing rate;
at a processing rate, processing the received EDA measurement signals to identify an EDA level category;
based on the identified EDA level category providing an appropriate signal, wherein:
   if the received EDA measurement is identified to be in a first EDA level category, providing an alarm indicating medical stress,
   if the received EDA measurement is identified to be in a second EDA level category, providing a signal indicating non-medical stress, and
   if the received EDA measurement is identified to be in a third EDA level category, providing a signal indicating that no stress is identified.

According to an aspect of some embodiments of the invention, there is also provided a device useful for providing information indicative of a reason for a stress situation in a human, comprising:
an electrodermal activity (EDA) sensor configured for determining electrodermal activity of a human at an EDA sensing rate and producing an EDA measurement signal related to the determined electrodermal activity; and
a processor functionally associated with the EDA sensor, configured to:
   receive from the EDA sensor an EDA measurement signal at an EDA sensing rate,
   at a processing rate, process the received EDA measurement signals to identify an EDA level category; and
   based on the identified EDA level category, provide an appropriate signal, wherein:
      if the received EDA measurement is identified to be in a first EDA level category, providing an alarm indicating medical stress,
      if the received EDA measurement is identified to be in a second EDA level category, providing a signal indicating non-medical stress, and
      if the received EDA measurement is identified to be in a third EDA level category, providing a signal indicating that no stress is identified.

In some embodiments, the alarm indicating medical stress is indicative of a medical stress situation selected from the group consisting of blood oxygenation stress, pulse rate stress, stress triggered by heart disease, stress triggered by kidney disease, stress triggered by improper processing of salts, and stress caused by epilepsy.

In some embodiments, the signal indicating non-medical stress is indicative of a non-medical stress situation selected from the group consisting of hunger, thirst, a dirty diaper, prolonged crying, gas in the digestive system, general discomfort, and pain.

Unless otherwise defined, all technical and scientific terms used herein have the same meaning as commonly understood by one of ordinary skill in the art to which the invention pertains. In case of conflict, the specification, including definitions, will take precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

As used herein, when a numerical value is preceded by the term “about”, the term “about” is intended to indicate +/−10%.

Embodiments of methods and/or devices of the invention may involve performing or completing selected tasks manually, automatically, or a combination thereof. Some embodiments of the invention are implemented with the use of components that comprise hardware, software, firmware or combinations thereof. In some embodiments, some components are general-purpose components such as general purpose computers or oscilloscopes. In some embodiments, some components are dedicated or custom components such as circuits, integrated circuits or software.

For example, in some embodiments, some of an embodiment is implemented as a plurality of software instructions executed by a data processor, for example which is part of a general-purpose or custom computer. In some embodiments, the data processor or computer comprises volatile memory for storing instructions and/or data and/or a non-volatile storage, for example, a magnetic hard-disk and/or removable media, for storing instructions and/or data. In some embodiments, implementation includes a network connection. In some embodiments, implementation includes a user interface, generally comprising one or more of input devices (e.g., allowing input of commands and/or parameters) and output devices (e.g., allowing reporting parameters of operation and results.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 is a pictorial illustration of an embodiment of a sensing unit forming part of a human monitoring device according to an embodiment of the teachings herein;

FIG. 2 is a pictorial illustration of an embodiment of a monitoring unit forming part of a human monitoring device according to an embodiment of the teachings herein;

FIGS. 3A, 3B, 3C, and 3D are schematic representations of an embodiment of a human monitoring device as used in a home setting according to an embodiment of the teachings herein, in a regular situation, in an emergency situation, in a low-battery situation, and in an insufficient signal situation, respectively;

FIG. 4 is a schematic representation of an embodiment of a human monitoring device as used in a medical facility setting according to an embodiment of the teachings herein;

FIG. 5 is a flow chart of an embodiment of a method for monitoring the health of a human according to an embodiment of the teachings herein;

FIGS. 6A-6C are pictorial illustrations of additional embodiments of a sensing unit forming part of a human monitoring device according to embodiments of the teachings herein; and

FIGS. 7A-7E are pictorial illustrations of additional embodiments of a monitoring unit forming part of a human monitoring device according to embodiments of the teachings herein.

### DESCRIPTION OF SOME EMBODIMENTS OF THE INVENTION

The invention, in some embodiments, relates to the field of sensors, and more particularly to the field of methods and devices for providing information useful in identifying a stress situation of a human, such as a baby or a disabled human.

In accordance with some embodiments of the teachings herein, there are provided methods for providing information useful in determining stress of a human, comprising:
positioning a pulse oximeter and an electrodermal activity (EDA) sensor to monitor a human;
at a processor functionally associated with the EDA sensor and the pulse oximeter, monitoring the human by receiving from the EDA sensor an EDA measurement signal at a EDA sensing rate;
at a processing rate, processing the received EDA measurement signals to identify an EDA indication of stress;
if an EDA indication of stress is identified:
   activating the pulse oximeter to determine a measure of at least one of pulse and blood oxygenation of the human;
   at the processor, receiving from the pulse oximeter at least one pulse oximeter signal related to the determined measure of the at least one of pulse and blood oxygenation;
   at the processor, processing the received pulse oximeter signal to identify a pulse oximeter indication of stress;
   if a pulse oximeter indication of stress is identified, the processor automatically providing an alarm signal.

In accordance with some embodiments of the teachings herein, there are also provided devices for monitoring a human for stress signals, comprising:
a pulse-oximeter configured for determining a measure of at least one of pulse and blood oxygenation of a human and producing a pulse oximeter signal related to the determined measure of at least one of pulse and blood oxygenation;
an electrodermal activity (EDA) sensor configured for determining electrodermal activity of a human at an EDA sensing rate and producing an EDA measurement signal related to the determined electrodermal activity;
a processor functionally associated with the pulse oximeter and with the EDA sensor; and
a wireless transmitter functionally associated with the processor,
wherein the processor is configured to:
   receive from the EDA sensor an EDA measurement signal at the EDA sensing rate,
   at a processing rate, process the received EDA measurement signals to identify an EDA indication of stress,
   if an EDA indication of stress is identified, activate the pulse oximeter to determine a measure of at least one of pulse and blood oxygenation of a human,
   receive from the pulse oximeter at least one pulse oximeter signal related to the determined measure of at least one of pulse and blood oxygenation,
   process the received the pulse oximeter signal to identify a pulse oximeter indication of stress, and
   if a pulse oximeter indication of stress is identified, automatically provide an alarm signal.

The principles, uses and implementations of the teachings herein may be better understood with reference to the accompanying description and figures. Upon perusal of the description and figures present herein, one skilled in the art is able to implement the invention without undue effort or experimentation.

Before explaining at least one embodiment of the invention in detail, it is to be understood that the invention is not limited in its applications to the details of construction and the arrangement of the components and/or methods set forth in the following description and/or illustrated in the drawings and/or the Examples. The invention can be implemented with other embodiments and can be practiced or carried out in various ways. It is also understood that the phraseology and terminology employed herein is for descriptive purpose and should not be regarded as limiting.

As discussed in the introduction, there is need for baby monitoring system which does not provide false alarms to the baby's caretaker, which has no hazardous cables required for activation, and which can function effectively for extended periods of time, for example at least 24 hours, at least 36 hours, or even up to 48 hours. There is need for similar systems for monitoring the health of elderly or disabled people, who are often not in a medical facility but require medical supervision.

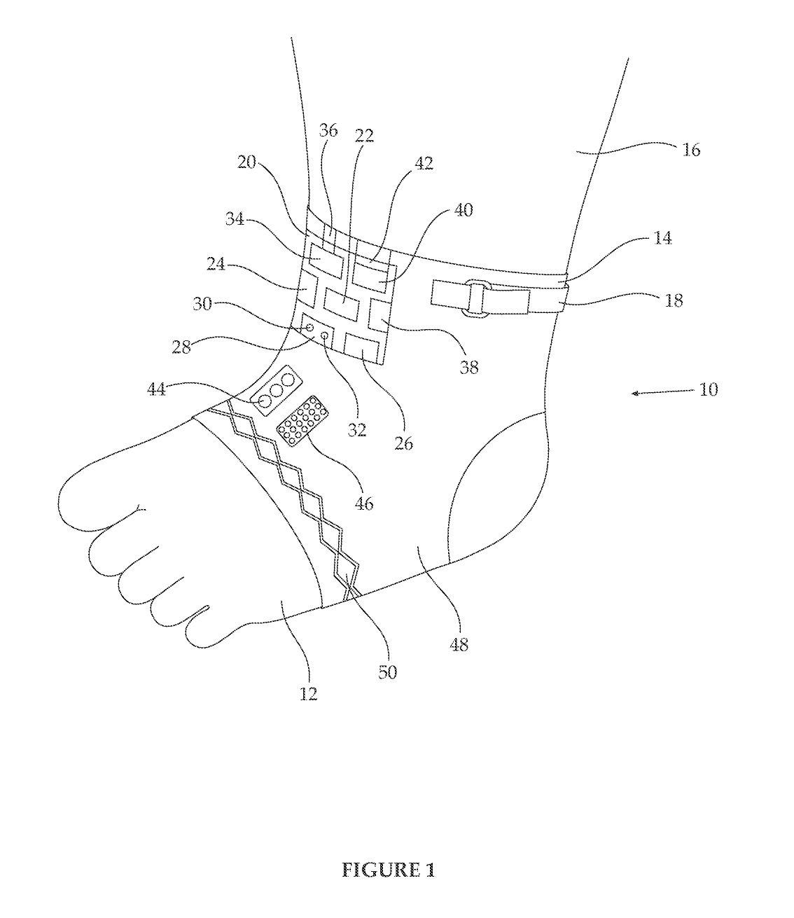

In the exemplary embodiments described hereinbelow with reference to FIGS. 1-7E, the monitoring system is used to monitor a baby, and the monitoring person is the baby's caregiver, such as a parent.

Reference is now made to FIG. 1, which is a pictorial illustration of an embodiment of a sensing unit forming part of a human monitoring device according to an embodiment of the teachings herein. As seen in FIG. 1, a sock 10 is placed around the baby's foot 12, such that an upper portion 14 of the sock 10 fits snugly around the baby's ankle 16 and cannot be moved by the baby.

In some embodiments, the upper portion 14 may be fitted to the size of ankle 16 by adjusting a width adjustor 18. In some embodiments, the width adjustor 18 informed of Velcro®. In some embodiments, the width adjustor 18 comprises at least one hook and multiple eyes, spaced around upper portion 14, and configured to receive the hook, in a similar structure to that of a bra strap. In some embodiments, the width adjustor 18 comprises a lever and multiple holes, spaced around upper portion 14, and configured to receive the lever, in a similar structure to that of a watch strap or a belt.

Upper portion 14 includes an electronics portion 20, located on an inner surface of the sock 10. Electronics portion 20 comprises a processor 22, such as an IC PIC MCU Flash processor commercially available from MicroChip Technology Inc. of Chandler, Ariz., USA, and a wireless transmitter 24, such as a Linx RF 433 MHz transmitter, commercially available from Linx Technologies of Merlin, Oreg., USA, functionally associated with a processor 22. In some embodiments, electronics portion 20 is enclosed in a pocket, separating the electronics from the baby's skin. In some embodiments, transmitter 24 is configured to transmit information using at least one wireless communication method selected from a group consisting of radio-frequency communication, Wi-Fi, GSM, and Bluetooth®.

Functionally associated with processor 22 is an electrodermal activity (EDA) sensor 26, such as an ag/agcl E203 disc electrode, commercially available from Warner Instruments LLC of Hamden, Conn., USA. EDA sensor 26 is mounted on sock 10 such that a sensing portion of the sensor is in direct contact with the baby's skin, and the signal processing portion of the sensor forms part of electronics portion 20. In some embodiments, the signal processing portion is located inside the electronics pocket, and the sensing portion is connected to the signal processing portion by a short wires (not shown) which are anchored to the sock 10. The EDA sensor 26 is configured to measure the baby's skin conductance at ankle 16 and to provide an EDA signal indicating the measured skin conductance to the processor 22 at a sensing rate. The sensing rate may be any suitable rate. That said, in some embodiments, the sensing rate is more frequently than every minute. In some embodiments the sensing rate is more frequently than every 30 seconds, 20 seconds, 10 seconds, or even more frequently than every second. In some embodiments, each EDA measurement has a set duration. In some embodiments, the duration is 15 seconds. In some embodiments, the duration is 30 seconds, 1 minute, or even up to five minutes.

Functionally associated with processor 22 is a pulse oximeter 28, such as a DCM02 or a DCM03, commercially available from APMKorea of Daejeon, KOREA. Pulse oximeter 28 is mounted on sock 10 such that a sensing portion, comprising at least one light source 30 and a photodetector 32 is in direct contact with the baby's skin, and the signal processing portion of the sensor forms part of electronics portion 20. In some embodiments, the pulse oximeter is enclosed in a protective enclosure (not shown), such as a silicon cushion, specifically designed to prevent sweat buildup on the pulse oximeter. In some embodiments, the signal processing portion is located inside the electronics pocket, and the light emitter and light receiver are connected to the signal processing portion by a short wires (not shown) which are anchored to the sock 10.

The pulse oximeter 28 is configured to measure the baby's pulse and blood oxygenation percentage at ankle 16 and to provide a pulse oximeter signal indicating at least one of, and preferably both the measured pulse and blood oxygenation percentage to the processor 22. The pulse oximeter 28 is also configured to be activated by a command from processor 22.

Unless activated by processor 22, pulse oximeter 28 is configured to remain in an inactive, energy-saving state. That said, in some embodiments, pulse oximeter 28 is periodically activated to obtain a pulse oximeter measurement in order to ensure that all is well with the baby being monitored.

Processor 22, transmitter 24, EDA sensor 26, and pulse oximeter 28, are all powered by an energy storage unit such as a battery 34 located in electronics portion 20. In some embodiments, battery 34 is a rechargeable battery such as a Lithium-Polymer prismatic 3.6V battery cell, commercially available from PowerStream Technologies of Orem, Utah, USA. In some embodiments, electronics portion 20 includes a charger port 36 for connecting a charger to enable charging of battery 34 while the battery is located in electronics portion 20 of sock 10. In some embodiments, battery 34 may be removed from sock 10, for example for recharging the battery on a suitable recharger (not shown).

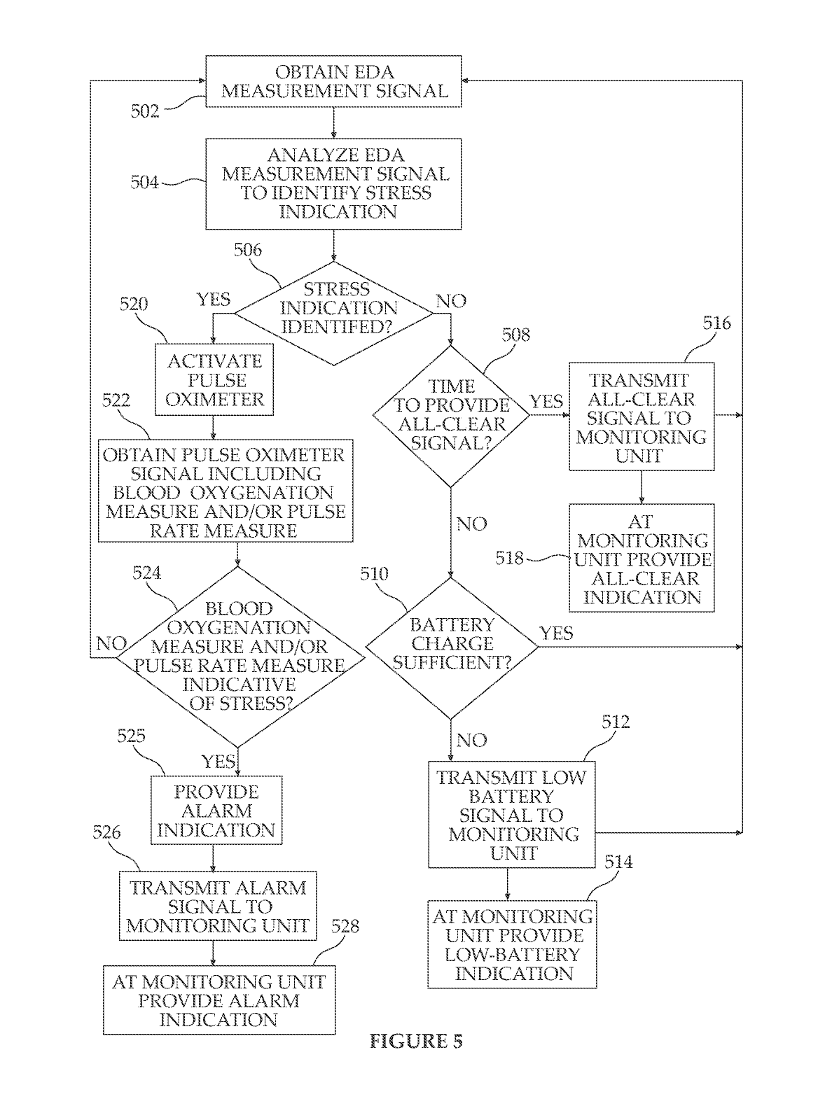

It is appreciated that when sock 10 is operated in accordance with the teachings herein, as described in further detail herein below with reference to FIG. 5, battery 34 is configured to provide sufficient power for continuous operation of the components of the sock 10 for a period of time of not less than 24 hours. In some embodiments, battery 34 is configured to provide sufficient power for continuous operation of the components of the sock 10 for a period of time of not less than 30 hours, 36 hours, or even up to 48 hours.

In some embodiments, at least one additional sensor 38, which is functionally associated with processor 22, is mounted on sock 10. In some embodiments, the sensor 38 includes a sensing portion which is positioned to be in direct contact with the baby, and a signal processing portion which forms part of electronics portion 20. In some embodiments, the entirety of sensor 38 is included in electronics portion 20. Additional sensor 38 may be any sensor suitable for monitoring the baby for a stress situation or for a health condition, such as, for example, a temperature sensor such as a SOT23 temperature probe commercially available from Texas Instruments Inc. of Dallas, Tex., USA, a motion sensor, a heart rate sensor, and a pulse sensor. In some embodiments, additional sensor 38 is powered by battery 34.

In some embodiments, electronics portion 20 includes a memory 40 functionally associated with EDA sensor 26 and/or with processor 22, for storing EDA levels measured by EDA sensor 26. The memory 40 may also store measurements provided by additional sensor 38. The memory 40 may store the EDA levels measured by EDA sensor 26 the sensing rate at which EDA sensor 26 receive the measurements, or at any other suitable rate, and may store the pulse and blood oxygenation measures measured by the pulse oximeter 28.

In some embodiments, memory 40 is functionally associated with transmitter 24, and is configured to wirelessly transmit stored information to a remote location, such as a nurse's station, a doctor's office, or a caregiver's mobile device.

In some embodiments, electronics portion 20 includes a port 42, such as a USB port, for connection of an external device or wire (not shown) to be functionally associated with processor 22 for downloading information from memory 40.

In some embodiments, memory 40 comprises a removable memory, configured to be removed from sock 10 for retrieval of the information contained in the memory. For example, the removable memory may be a USB flash drive or a SD card.

In some embodiments, sock 10 further comprises a visual indicator 44, such as a source of visible light, for example a Bicolor SMT LED commercially available from Lite-On Inc. of Milpitas, Calif., USA, and/or an aural indicator 46, such as a Buzzer SMD speaker commercially available from Sanco Electronics Co. Ltd. of Ningbo, CHINA. Visual indicator 44 and aural indicator 46 may form part of electronics portion 20, and are configured to provide an alarm indication perceivable to a human, such as a visual alarm or an audible alarm, if a stress situation is identified by processor 22, as described in further detail hereinbelow with reference to FIG. 5.

In some embodiments, an external surface 48 of sock 10 include one or more decorative elements 50. Decorative elements 50 may be any suitable decorative elements, and may have any suitable colors. In some embodiments, decorative elements 50 are geometrical shapes colored in black white and red, which are known to stimulate a neonate's eyesight. In some embodiments, decorative elements 50 comprised images, such as images of the characters which would attract a child's attention.

In some embodiments, decorative elements 50 may comprise a rattle or a toy sewn on to sock 10 for the baby to play with while sock 10 is on his foot. However, for EDA sensor 26 and pulse oximeter 28 to function correctly, it is important that sock 10 remain properly positioned on the baby's foot. Therefore it is important that the baby should not be able to move the sock 10 on his foot while playing the decorative elements 50.

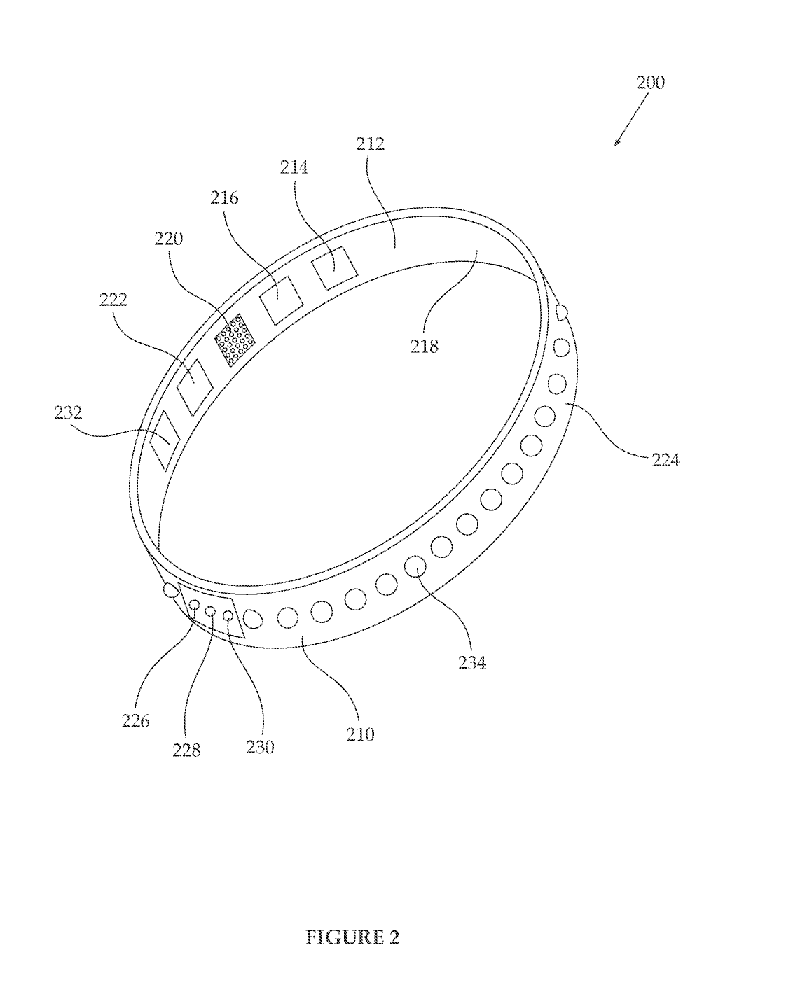

Reference is now made to FIG. 2, which is a pictorial illustration of an embodiment of a monitoring unit forming part of a baby monitoring device according to an embodiment of the teachings herein. As seen in FIG. 2, a monitoring unit 200 comprises a bracelet 210 suitable to be worn around the wrist of a human, typically the baby's parent or caregiver. Bracelet 210 may be formed of any suitable material such as silicon, fabrics, leather, or any other suitable material. In some embodiments, bracelet 210 comprises a slip-on bracelet, configured to be slipped over a wearer's hand and onto the wearer's wrist. In some embodiments, bracelet 210 is openable, and comprises a size adjustor (not shown) for adjusting the size of bracelet 210 to the size of the wearer's wrist. The size adjustor may be any suitable adjustor, such as a Velcro strap or a silicon strap.

Formed on an inner surface 212 of bracelet 210 is a receiver 214, such as a Linx RF 433 MHz receiver, commercially available from Linx Technologies of Merlin, Oreg., USA, configured to receive signals from a transmitter of a sensing unit, such as transmitter 24 of the sock 10 of FIG. 1. In some embodiments, receiver 214 is configured to receive the signal transmitted using at least one wireless communication method selected from a group consisting of radio-frequency communication, Wi-Fi, GSM, and Bluetooth®.

Functionally associated with receiver 214 is a processor 216, such as an IC PIC MCU Flash processor commercially available from MicroChip Technology Inc. of Chandler, Ariz., configured to process signals received by receiver 214.

Functionally associated with processor 216 are a visual indicator 218, such as a Bicolor SMT LED commercially available from Lite-On Inc. of Milpitas, Calif., USA, an aural indicator 220, such as a Buzzer SMD speaker commercially available from Sanco Electronics Co. Ltd. of Ningbo, CHINA, and a tactile indicator 222, such as a vibration motor 3, commercially available from Precision Microdrives Ltd. of London, UK.

Visual indicator 218 is formed on an outer surface 224 of bracelet 210 and may be any suitable visual indicator emitting light perceivable by a human, such as the one or more LED lights. In some embodiments, visual indicator 218 comprises a red LED light 226 and a green LED light 228. In some embodiments, the visual indicator 218 also comprises an orange LED light 230. In some such embodiments, each of LED lights 226, 228, and 230 may be used to provide indication to a wearer of bracelet 210 in different situations as described hereinbelow.

Aural indicator 220 may be any suitable aural indicator providing an indication perceivable by a human, such as a speaker, and may be formed on inner surface 212 or on outer surface 224 of bracelet 210. In some embodiments, aural indicator 220 is configured to provide at least two different audible indications, which are perceivable by a human and distinguishable by a human. For example, the different audible indications may have different amplitudes, different frequencies, or different patterns. In some such embodiments, each of the different audible indication may be used to provide indication to a wearer of bracelet 210 in different situations as described hereinbelow.

Tactile indicator 222 may be any suitable tactile indicator providing an indication perceivable by a human, such as a small piezoelectric speaker as known in the art of cellular telephony, and it typically formed on inner surface 212 of bracelet 210. In some embodiments, tactile indicator 222 is configured to provide at least two different tactile indications, which are perceivable by a human and distinguishable by a human. For example, the different tactile indications may have different frequencies or different amplitudes. In some such embodiments, each of the different tactile indications may be used to provide indication to a wearer of bracelet 210 in different situations as described hereinbelow.

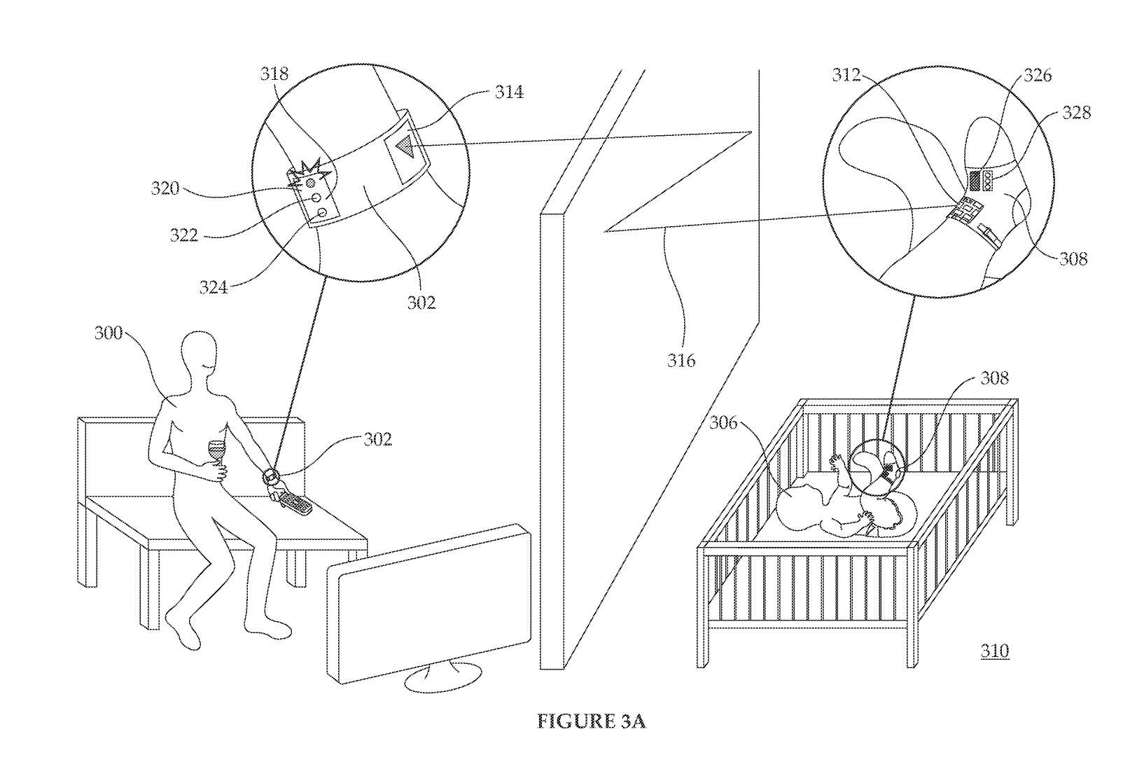

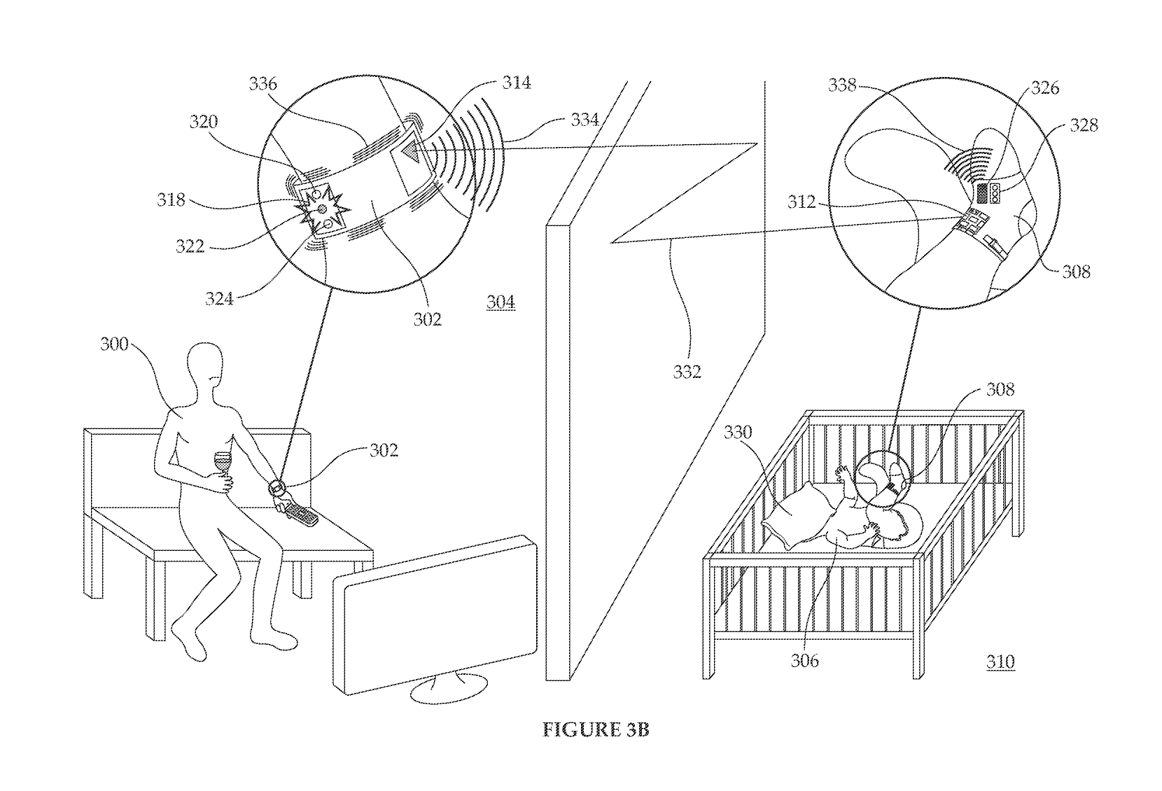

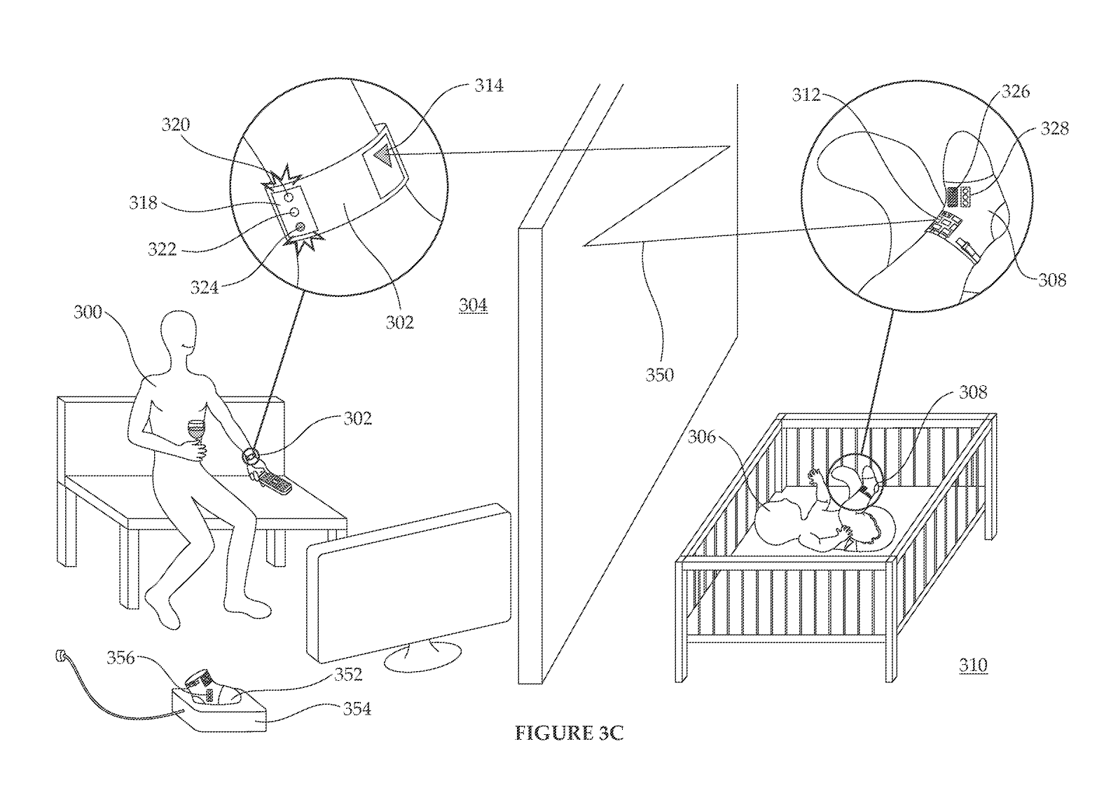

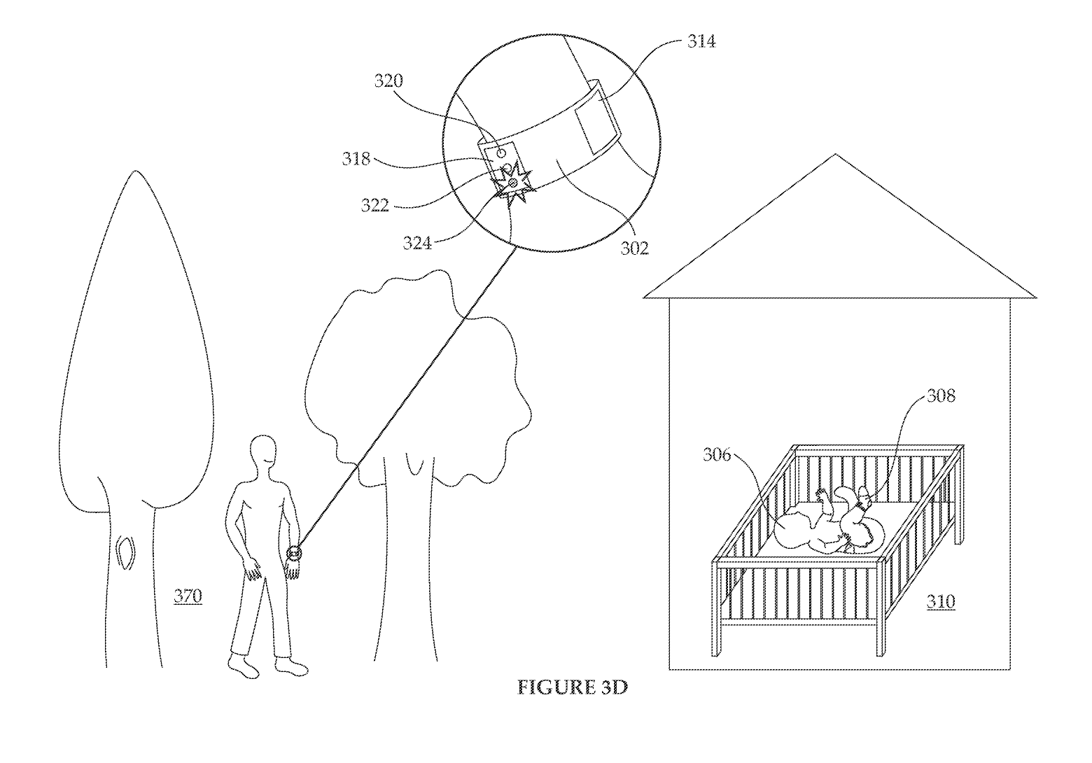

As will be described in further detail hereinbelow in reference to FIGS. 3A and 3B, in response to receipt of the signal from receiver 214, processor 216 activates at least one of visual indicator 218, aural indicator 220, and tactile indicator 222, in order to provide a human-perceivable indication of the baby's health status and/or of system status to the wearer of bracelet 210.

In some embodiments, processor 216 is configured to activate some or all of visual indicator 218, aural indicator 220, and tactile indicator 222 in response to a signal received by receiver 214. For example, processor 216 activates visual indicator 218 to emit a red light from red LED light 226, aural indicator 220 to sound alarm sounds, and tactile indicator 222 to vibrate, in response to an alarm signal received by receiver 214 from the sensing unit.

In some such embodiments, processor 216 is configured to distinguish between different signals received by receiver 214 by activating some or all of visual indicator 218, aural indicator 220, and tactile indicator 222 to provide different indications to the user in response to such different signals. For example, processor 216 may activate visual indicator 218 to emit a red light from red LED light 226 in response to an alarm signal received by receiver 214 and to emit a green light from green LED light 228 in response to an all-clear signal received by receiver 214. As another example, processor 216 may activate visual indicator 218 to emit a flashing light in response to an alarm signal received by receiver 214 and to emit a continuous light in response to an all-clear signal received by receiver 214. In a similar manner, processor 216 may activate aural indicator 220 and/or tactile indicator 222 to provide a high-frequency indication in response to an alarm signal received by receiver 214 and to provide low-frequency indication in response to an all-clear signal received by receiver 214.

In some embodiments, processor 216 is configured to activate a different one or a different subset of visual indicator 218, aural indicator 220, and tactile indicator 222 in response to different signals received by receiver 214. For example, processor 216 may activate visual indicator 218 in response to an all-clear signal received by receiver 214, indicating that the baby is healthy and safe, and may activate all of visual indicator 218, tactile indicator 222 and aural indicator 220 in response to an alarm signal received by receiver 214.

In some embodiments, processor 216 is configured to activate at least one of the visual indicator 218, aural indicator 220, and tactile indicator 222, in order to indicate a change in system status. For example, processor 216 may activate visual indicator 218 to flash an orange light from LED light 230 in response to a low-battery signal received by receiver 214, or in response to receiving insufficient signals or to being out of range of the sensing unit of FIG. 1.

Receiver 214, processor 216, visual indicator 218, aural indicator 220, and tactile indicator 222, are all powered by a battery 232 formed on inner surface 212 of bracelet 210.

In some embodiments, battery 232 is a rechargeable battery such as a Lithium-Polymer prismatic 3.6V battery cell, commercially available from PowerStream Technologies of Orem, Utah, USA. In some embodiments, bracelet 210 includes a charger port (not shown) for connecting a charger to enable charging of battery 232 while the battery is located in bracelet 210. In some embodiments, battery 232 may be removed from bracelet 210, for example for recharging the battery on a suitable recharger (not shown).

In some embodiments, battery 232 is configured to provide sufficient power for continuous operation of components of bracelet 210 for a period of time of not less than 24 hours. In some embodiments, battery 232 is configured to provide sufficient power for continuous operation of the components of bracelet 210 for a period of time of not less than 26 hours, 36 hours, or even up to 48.

In some embodiments, processor 216 occasionally, periodically, or intermittently, checks the charge of battery 232, and notifies the wearer of bracelet 210 if battery 232 needs to be recharged. Such notification may be achieved, for example, by activating one or more visual indicator 218, aural indicator 220, and tactile indicator 222 to provide a unique indication signal in a low battery in the monitoring unit. For example, processor 216 may activate orange LED light 230 of visual indicator 218 to flash as indication of low charge on battery 232. As another example, processor 216 may activate all of LED light 226, 228, and 230 of the visual indicator 218 as indication of low charge on battery 232.

In some embodiments, outer surface 224 of bracelet 210 has a decorative appearance, for example including a string of beads 234, though any other decorative element is considered within the scope of the teachings herein. It is appreciated that in some situation the baby's caregiver's would want to be just more formally, such as at weddings, funerals, and other events. The decorative appearance of outer surface 232 of bracelet 210, which may have the appearance of a piece of jewelry while maintaining its functionality, enables the caregiver to continue wearing bracelet 210 in such formal occasions and to continually be aware of the well-being of the baby.

Reference is now made to FIGS. 3A, 3B, 3C, and 3D, which are schematic representations of an embodiment of a baby monitoring device as used in a home setting according to an embodiment of the teachings herein, in a regular situation, in an emergency situation, in a low-battery situation, and in an out-of-range situation, respectively.

As seen in FIG. 3A, a caregiver 300 wearing a monitoring unit 302, such as bracelet 210 of FIG. 2, is located in a first room 304, such as a kitchen, living room, bedroom, or any other location. The caregiver may be involved in any activity, and is shown in FIG. 3A as relaxing while watching television and drinking a glass of wine. A baby 306 wearing a sensing unit 308, such as sock 10 of FIG. 1, is located in a second room 310, which is in wireless communication range of first room 304.

As described herein below with reference to FIG. 5, sensing unit 308 checks the electrodermal activity of baby 306 at an EDA sensing rate using an EDA sensor (not shown) such as EDA sensor 26 of FIG. 1. As long as the electrodermal activity of baby 306 does not indicate any stress condition, the sensing unit 308 does not check the baby's blood saturation level and does not provide an alarm signal to caregiver 300.

Occasionally, transmitter 312 of sensing unit 308, similar to transmitter 24 of FIG. 1, wirelessly transmits an all-clear signal which is received by receiver 314 of monitoring unit 302, similar to receiver 214 of FIG. 2, as indicated by arrow 316. In response to receipt of an all-clear signal from sensing unit 308, monitoring unit 302 provides an indication, perceivable by caregiver 300, of the baby's well-being. In some embodiments, in response to transmission of an all-clear signal, sensing unit 308 provides an indication, perceivable to a human, of the baby's well-being, such as by providing a visual indication using visual indicator 326, similar to visual indicator 44 of FIG. 1, or by providing an audible indication using aural indicator 328, similar to aural indicator 46 of FIG. 1.

In the illustrated example, monitoring unit 302 includes a visual indicator 318 comprising three LED lights: a green LED light 320, a red LED light 322, and orange LED light 324. In response to receipt of the all clear signal, the processor (not shown) of monitoring unit 302 activates visual indicator 318 to flash a green light from LED light 320, thereby indicating that the caregiver 300 that all is well with the baby and there is no need to go check on baby 306. However, any suitable indication, such as an aural indication, tactile indication, visual indication, or any combination thereof, may be provided to the baby's caregiver 300, as long as the baby's caregiver can distinguish the indication from an alarm indication or system problem indication and can identify that the indication means that all is well with the baby.

Transmitter 312 may transmit an all-clear signal to receiver 314 intermittently or periodically. In some embodiments, an all-clear signal is transmitted every set period of time, for example once, every 15 minutes, once every 30 minutes, once an hour, or even once in two hours. In some embodiments, an all-clear signal is transmitted after a set number of EDA measurements since the transmission of the last all-clear signal or of an alarm signal, for example after 20 EDA measurements.

Turning to FIG. 3B, the caregiver 300 is an in first room 304, while in second room 310 baby 306 is sleeping with a pillow 330 on his face. As described herein below with reference to FIG. 5, sensing unit 308 checks the electrodermal activity of baby 306 at an EDA sensing rate using the EDA sensor and upon sensing an increase in the baby's electrodermal activity, a processor (not shown) of sensing unit 308 activates a pulse oximeter (not shown), similar to pulse oximeter 26 of FIG. 1.

As explained hereinbelow with reference to FIG. 5, if one or more measurements by the pulse oximeter are indicative of stress, such as the blood oxygenation portion of the measurement being below a predetermined blood oxygenation threshold and/or the pulse rate portion of the measurement being below a low pulse rate threshold or above a high pulse rate threshold, transmitter 312 of sensing unit 308 wirelessly transmits an alarm signal which is received by receiver 314 of monitoring unit 302 as indicated by arrow 332. In some embodiments, the alarm signal transmitted by sensing unit 308 is indicative of the cause for alarm, such as a problem with blood oxygenation or a problem with the baby's pulse.

In response to receipt of an alarm signal from sensing unit 308, monitoring unit 302 provides an alarm indication, perceivable by caregiver 300.

In the illustrated example, monitoring unit 302 includes, in addition to visual indicator 318, an aural indicator (not shown), and a tactile indicator (not shown). In response to receipt of the alarm signal, the processor of monitoring unit 302 activates visual indicator 318 to flash a red light from LED light 322, sound an alarm from the aural indicator as shown at reference 334, and cause the tactile indicator to vibrate as shown at reference 336, thereby notifying the caregiver 300 of the emergency situation and alerting the caregiver to respond.

However, any suitable indication, such as an aural indication, tactile indication, visual indication, or any combination thereof, may be provided to the baby's caregiver 300, as long as the baby's caregiver is alerted to respond to the baby's stress situation.

In the illustrated example, in addition to providing an alarm indication at monitoring unit 302, an alarm indication perceivable to a human is also provided at sensing unit 308. As seen, upon identifying a stress indicator, the processor of sensing unit 308 activates visual indicator 326 to flash a red light, and activates aural indicator 328 to sound an audible alarm, as indicated by reference numeral 338.

As seen in FIG. 3C, caregiver 300 wearing monitoring unit 302 is located in first room 304, while baby 306 wearing a sensing unit 308 is located in a second room 310, which is in wireless communication range of first room 304.

Occasionally, a processor (not shown) of sensing unit 308 checks the charge of a battery (not shown) powering elements of sensing unit 308, similar to battery 34 of FIG. 1. If a low charge is detected in the battery of sensing unit 308, transmitter 312 of sensing unit 308 wirelessly transmits a low-battery signal which is received by receiver 314 of monitoring unit 302, as indicated by arrow 350. In response to receipt of a low-battery signal from sensing unit 308, monitoring unit 302 provides an indication, perceivable by caregiver 300, of the status of the battery of sensing unit 308.

In the illustrated example, in response to receipt of the low-battery signal, the processor of monitoring unit 302 activates visual indicator 318 to flash an orange light from LED light 324, thereby indicating that the caregiver 300 that the battery of sensing unit 308 needs to be recharged. However, any suitable indication, such as an aural indication, tactile indication, visual indication, or any combination thereof, may be provided to the baby's caregiver 300, as long as the baby's caregiver can distinguish the indication from other indications such as an alarm indication, and all-clear indication, and an out-of-range indication.

In the illustrated embodiment, caregiver 300 has a second sensing unit 352 being charged using the recharger 354 during use of sensing unit 308. Thus, in response to noticing the low-battery indication, caregiver 300 may remove sensing unit 352 from recharger 354 and place it on the foot of baby 306 instead of sensing unit 308. Subsequently, caregiver 300 may connect to sensing unit 308 to recharger 354 for recharging of the battery of sensing unit 308. In this way, caregiver 300 can make sure that the EDA level, pulse, and blood oxygenation of baby 306 are constantly monitored, even during recharging of sensing unit 308.

In some embodiments, the sensing unit, such as sensing unit 308 or sensing unit 352, or the recharger, such as recharger 354, provide an indication to the user, such as a visual indication or an audible indication, indicating that the sensing unit located in the recharger is currently being charged. In the illustrated example, a visual indicator 356, similar to visual indicator 44 of FIG. 1, emits a continuous orange light to indicate that the sensing unit 352 is fully charged. In some embodiments, during recharging of sensing unit 352, and prior to it being fully charged, visual indicator 356 flashes an orange light to indicate current charging.

Turning to FIG. 3D, it is seen that caregiver 300 wearing monitoring unit 302 is now located in a yard 370, while baby 306 wearing sensing unit 308 remains in room 310. However, yard 370 is not in wireless communication range of room 310.

Occasionally, a processor (not shown) of monitoring unit 302 checks the signal quality of signals received from sensing unit 308. If the processor of monitoring unit 300 detects that the signal quality is insufficient, monitoring unit 302 provides an indication, perceivable by caregiver 300, that the monitoring unit 302 is receiving insufficient signals. Such insufficient signals may be received, for example, when the monitoring unit 302 if out of communication range from sensing unit 308, as in the illustrated example.

In the illustrated example, in response to detection of insufficient signal quality, the processor of monitoring unit 302 activates visual indicator 318 to emit a continuous beam of orange light from LED light 324, thereby indicating that the caregiver 300 that the monitoring unit is receiving signals of insufficient quality and is probably out of range, and will not receive an alarm signal if such is provided by sensing unit 308. However, any suitable indication, such as an aural indication, tactile indication, visual indication, or any combination thereof, may be provided to the baby's caregiver 300, as long as the baby's caregiver can distinguish the indication from other indications such as an alarm indication, and all-clear indication, and a low-battery indication.

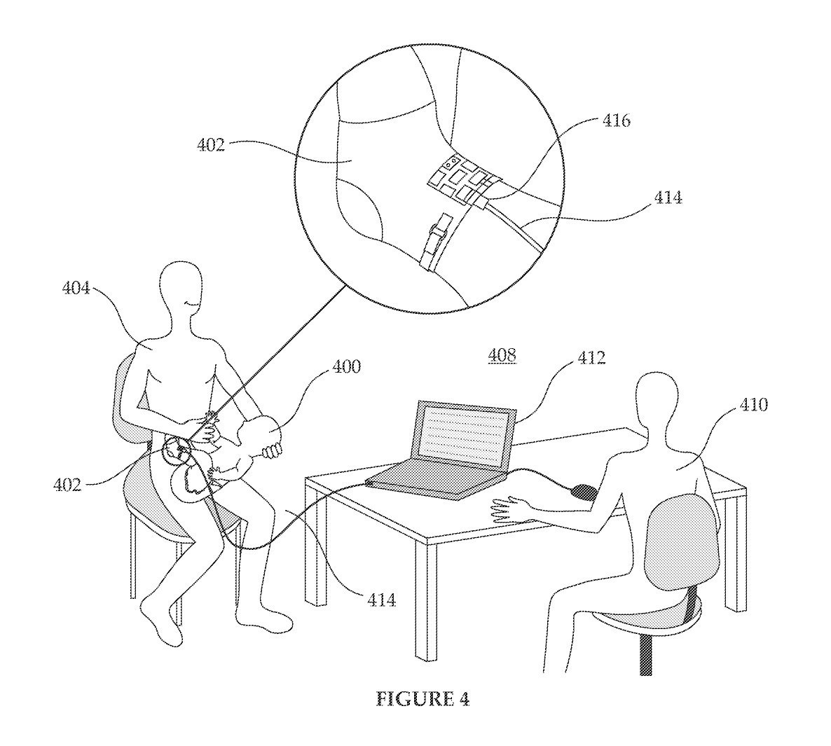

Reference is now made to FIG. 4, which is a schematic representation of an embodiment of a baby monitoring device as used in a medical facility setting according to an embodiment of the teachings herein.

As seen, a baby 400 wearing a sensing unit 402 is located in a medical facility such as a doctor's office 408. A member of the medical staff 410, such as a doctor or a nurse, retrieves the information stored in a memory (not shown) of sensing unit 402, such as memory 40 of FIG. 1.

In the illustrated embodiment, the data is retrieved by connecting sensing unit 402 to a computer 412. Specifically, in the illustrated embodiment, a double sided USB wire 414 is connected to a suitable USB port in computer 412 and to a USB port 416, similar to port 42 of FIG. 1, in sensing unit 402. The medical staff member 410 can thus transfer files and medical data collected in the memory of sensing unit 402 to the computer 412 for purposes of monitoring, diagnosis, or any other suitable medical needs.

It is appreciated that the medical data may be retrieved from the memory of sensing unit 402 in any suitable way. In some embodiments, the memory is removed from the sensing unit 402 and is connected to an appropriate device for accessing the data thereon, such as by connecting a memory in the form of a USB flash drive memory to the computer 412 or by connecting the memory in the form of a SD card to a suitable card reader. In some embodiments, the data contained in the memory of sensing unit 402 is transmitted from sensing unit 402 directly to computer 412 via a transmitter (not shown) of sensing unit 402 and via a suitable network.

Reference is now made to FIG. 5, which is a flow chart of an embodiment of a method for monitoring the health of a baby according to an embodiment of the teachings herein.

As seen in FIG. 5, a sensing unit, such as sock 10 of FIG. 1, employs an EDA sensor such as EDA sensor 26 of FIG. 1, to obtain an EDA measurement signal at a sensing rate, as indicated at reference numeral 502.

The sensing rate may be any suitable sensing rate. That said, in some embodiments, the sensing rate is more frequently than every minute. In some embodiments the sensing rate is more frequently than every 30 seconds, 10 seconds, 5 seconds, or even as frequently as every second. In some embodiments, each EDA measurement has a set duration. In some embodiments, the duration is 15 seconds. In some embodiments, the duration is 30 seconds, 1 minute, or even up to five minutes.

A processor of the sensing unit, similar to processor 22 of FIG. 1, analyzes the EDA measurement signal to identify an EDA stress indication at reference numeral 504. The EDA stress indication may be any suitable EDA indication. That said, in some embodiments, the indication comprises a single EDA measurement being above an EDA threshold.

In some embodiments, the indication comprises a function of multiple EDA measurements being above the EDA threshold. In some such embodiments, the function is an average of multiple EDA measurement. In some such embodiments, the function is a mean of multiple EDA measurements. The EDA threshold may be any suitable threshold. That said, in some embodiments, the EDA threshold is 37% of phasic change over the tonic level. In some embodiments, the EDA threshold is 40%, 45% or even greater than 50% change over the tonic level.

As seen in reference 506, if the processor of the sensing unit did not identify an EDA stress indication, the processor determines whether it is time to provide an all-clear signal to the monitoring unit, as seen at reference 508. If it is not time to provide an all-clear signal to the monitoring unit, the processor checks the charge of the battery powering the sensing unit, similar to battery 34 of FIG. 1, as seen at reference 510.

If the battery charge is sufficient, the processor continues to obtain EDA measurement signals as seen at reference 502. If however, as seen at reference 512, the battery charge is not sufficient, the processor of the sensing unit employs a transmitter of the sensing unit, similar to transmitter 24 of FIG. 1, to transmit a low-battery signal to the monitoring unit. Subsequently, the processor of the sensing unit continues to obtain EDA measurement signal as seen at reference numeral 502. Concurrently, as seen at reference 514, upon receipt of the low-battery signal from the sensing unit, the monitoring unit provides a low-battery indication to the user, as described hereinabove with reference to FIG. 3C.

Returning to reference 508, if it is time to provide an all-clear signal, the processor of the sensing unit employs the transmitter of the sensing unit to transmit an all-clear signal to the monitoring unit, as seen at reference 516. Subsequently, the processor of the sensing unit continues to obtain EDA measurement signal as seen at reference numeral 502. Concurrently, as seen at reference 518, upon receipt of the all-clear signal from the sensing unit, the monitoring unit provides an all-clear indication to the user, as described hereinabove with reference to FIG. 3A.

Returning to reference 506, if the processor identified an EDA stress indication, it proceeds to activate a pulse oximeter of the sensing unit, similar to pulse oximeter 28 of FIG. 1, from an inactive, energy-saving state, to determine at least one of, preferably both a pulse of the baby and a blood oxygenation percentage of the baby, as seen at reference 520. The processor of the sensing unit obtains a pulse oximeter signal from the pulse oximeter, as seen in reference 522, and analyzes the pulse oximeter signal to determine whether the blood oxygenation measure of the signal is indicative of stress and/or whether the pulse rate measure of the signal is indicative of stress, as seen at reference 524.

In some embodiments, the pulse oximeter signal comprises a single pulse measurement by the pulse oximeter. In some embodiments, the pulse oximeter signal comprises a single blood oxygenation measurement by the pulse oximeter. In some embodiments, the pulse oximeter signal comprises a single measurement by the pulse oximeter including pulse measurement and blood oxygenation measurement.

In some embodiments, the pulse oximeter signal comprises a function of multiple measurements by the pulse oximeter. In some such embodiments, the function is an average of the multiple measurements by the pulse oximeter. In some such embodiments, the function is a mean of the multiple measurements by the pulse oximeter. In some embodiments, the pulse oximeter signal comprises multiple pulse oximeter signal, each representing a single measurement by the pulse oximeter.

The pulse oximeter indication of stress may be any suitable pulse oximeter indication. In some embodiments, the pulse oximeter indication comprises the blood oxygenation measure of the pulse oximeter signal being below a blood oxygenation threshold. In some embodiments, the pulse oximeter indication comprises the blood oxygenation measures of at least two pulse oximeter signals being below the blood oxygenation threshold.

The blood oxygenation threshold can be any suitable threshold indicative of baby stress. That said, in some embodiments, the blood oxygenation threshold is 60% saturation. In some embodiments, the blood oxygenation threshold is 70% saturation, 80% saturation, or even 92% saturation.

In some embodiments, the pulse oximeter indication comprises the pulse rate measure of the pulse oximeter signal being below a low pulse rate threshold. In some embodiments, the pulse oximeter indication comprises the pulse rate measures of at least two pulse oximeter signals being below the low pulse rate threshold.

The low pulse rate threshold can be any suitable threshold indicative of baby stress. That said, in some embodiments, the low pulse rate threshold is 50 beats per minute. In some embodiments the pulse rate threshold is 45 beats per minute.

In some embodiments, the pulse oximeter indication comprises a pulse rate portion of the pulse oximeter signal being above a high pulse rate threshold. In some embodiments, the pulse oximeter indication comprises the pulse rate measures of at least two pulse oximeter signals being above the high pulse rate threshold.

The high pulse rate threshold can be any suitable threshold indicative of baby stress. In some embodiments, the high pulse rate threshold is 140 beats per minute. In some embodiments the high pulse rate threshold is 145, 150, or even 160 beats per minute.

If the pulse oximeter signal is not indicative of stress, the processor of the sensing unit continues to obtain EDA measurement signals as seen at reference 502. If however, as seen at reference 525, an indication of stress is identified in the pulse oximeter signal, the processor of the sensing unit provides an alarm indication perceivable to a human, such as by activation of the visual indicator of the sensing unit, or by activation of the aural indicator of the sensing unit. As seen at reference 526, the processor also employs the transmitter of the sensing unit to transmit an alarm signal to the monitoring unit. Upon receipt of the alarm signal from the sensing unit, the monitoring unit provides an alarm indication to the user, as described hereinabove with reference to FIG. 3B, as seen at reference 528.

It is appreciated that activation of the pulse oximeter only upon receipt of a stress indication from the EDA measurement signal greatly reduces the battery consumption of the sensing unit, and facilitates the battery's ability to last for more than 24 hours nonstop. On the other hand, the regular EDA measurements have only a short time elapsing between one measurement and another, thereby making sure the baby stress signals don't go on identified.

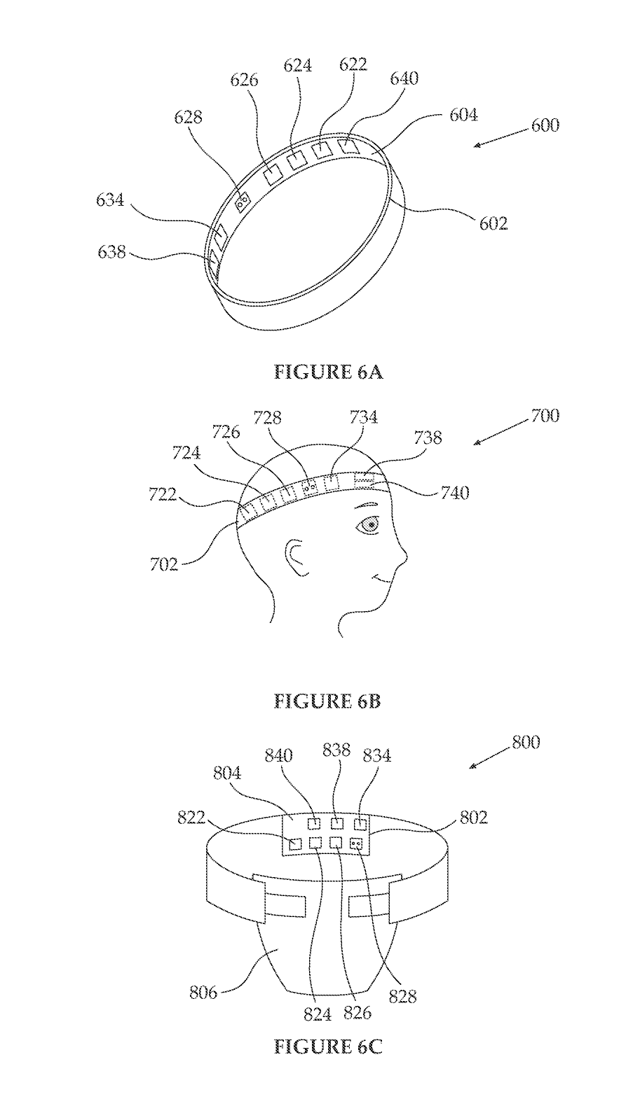

Reference is now made to FIGS. 6A-6C, which are pictorial illustrations of additional embodiments of a sensing unit forming part of a baby monitoring device according to embodiments of the teachings herein.

As seen in FIG. 6A, a sensing unit 600 comprises a bracelet 602 having an inner surface 604, is configured to be placed on a baby's wrist. Bracelet 602 may be formed of any suitable material such as silicon, fabrics, leather, or any other suitable material.

In some embodiments, bracelet 602 comprises an elasticated slip-on bracelet, configured to be slipped over a wearer's hand and onto the wearer's wrist and then to fit snugly around the wrist. In some embodiments, bracelet 602 is openable, and comprises a size adjustor (not shown) for adjusting the size of bracelet 602 to the size of the baby's wrist. The size adjustor may be any suitable adjustor, such as a Velcro strap, a watch strap, or a spring based portion forming part of bracelet 602.

Mounted onto inner surface 604 are a processor 622, a transmitter 624, an EDA sensor 626, a pulse oximeter 628, and the battery 634, all configured and operative as described for equivalent parts hereinabove with reference to FIG. 1. Bracelet 602 may also include one or more additional sensors 638 and a memory 640, configured and operative described hereinabove with reference to FIG. 1. In use, EDA sensor 626 and pulse oximeter 628 engage the baby's skin, and are formed such that they will not be reached by ambient light, for example by bracelet 602 being formed of an opaque material.

Turning to FIG. 6B, it is seen that a sensing unit 700 comprises an opaque headband 702 having an inner surface 704, and configured to be placed around a baby's head. Headband 702 may be formed of any suitable material such as silicon, fabrics, leather, or any other suitable material.

In some embodiments, headband 702 comprises an elasticated slip-on headband, configured to be slipped over a baby's head and then to fit snugly around the baby's head touching the forehead. In some embodiments, headband 702 is openable, and comprises a size adjustor (not shown) for adjusting the size of headband 702 to the size of the baby's head. The size adjustor may be any suitable adjustor, such as a Velcro strap, a watch strap, or a spring based portion forming part of headband 702.

Mounted onto inner surface 704 are a processor 722, a transmitter 724, an EDA sensor 726, a pulse oximeter 728, and the battery 734, all configured and operative as described for equivalent parts hereinabove with reference to FIG. 1. Headband 702 may also include one or more additional sensors 738 and a memory 740, configured and operative described hereinabove with reference to FIG. 1.

In use, EDA sensor 726 and pulse oximeter 728 engage the baby's skin, and are formed such that they will not be reached by ambient light, for example by headband 702 being formed of an opaque material. It is appreciated that a band, similar to headband 702, may be places at any suitable location around the baby's body, such as on the torso, thigh, or arm.

Turning to FIG. 6C, it is seen that a sensing unit 800 comprises a diaper insert 802 having an inner surface 804 and configured to be fitted onto, or clipped onto, a baby's diaper 806, such that inner surface 804 engages the baby's skin.

Mounted onto inner surface 804 are a processor 822, a transmitter 824, an EDA sensor 826, a pulse oximeter 828, and a battery 834, all configured and operative as described for equivalent parts hereinabove with reference to FIG. 1. Diaper insert 802 may also include one or more additional sensors 838 and a memory 840, configured and operative described hereinabove with reference to FIG. 1. In use, EDA sensor 826 and pulse oximeter 828 engage the baby's skin, and are formed such that they will not be reached by ambient light.

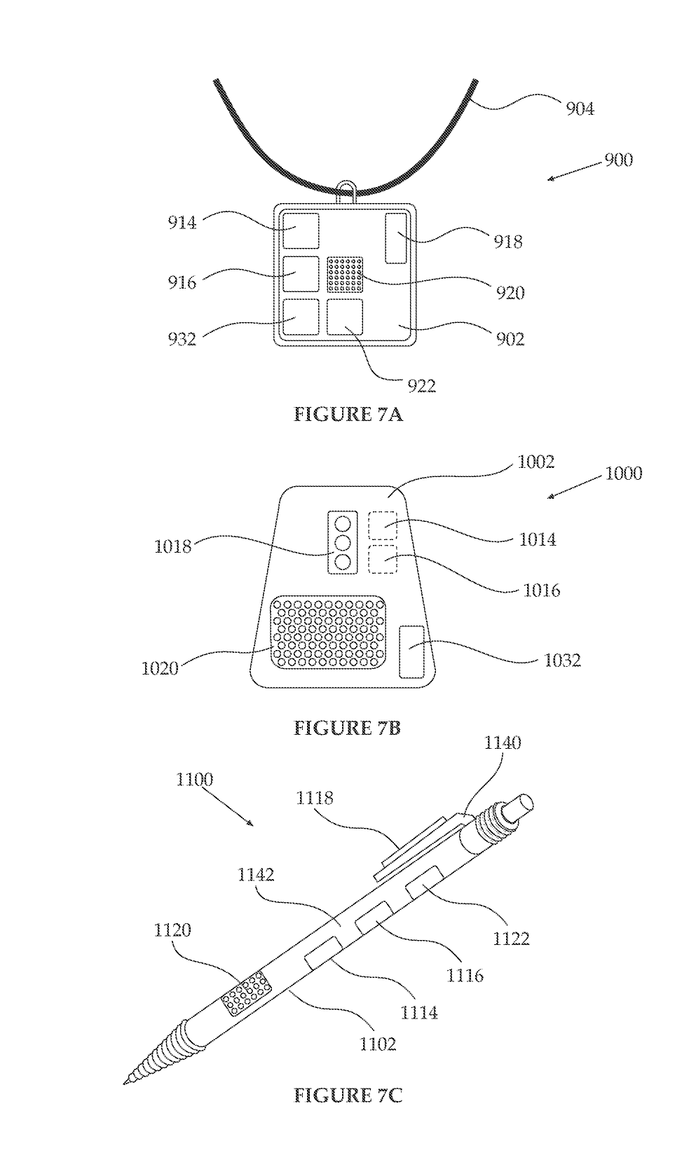

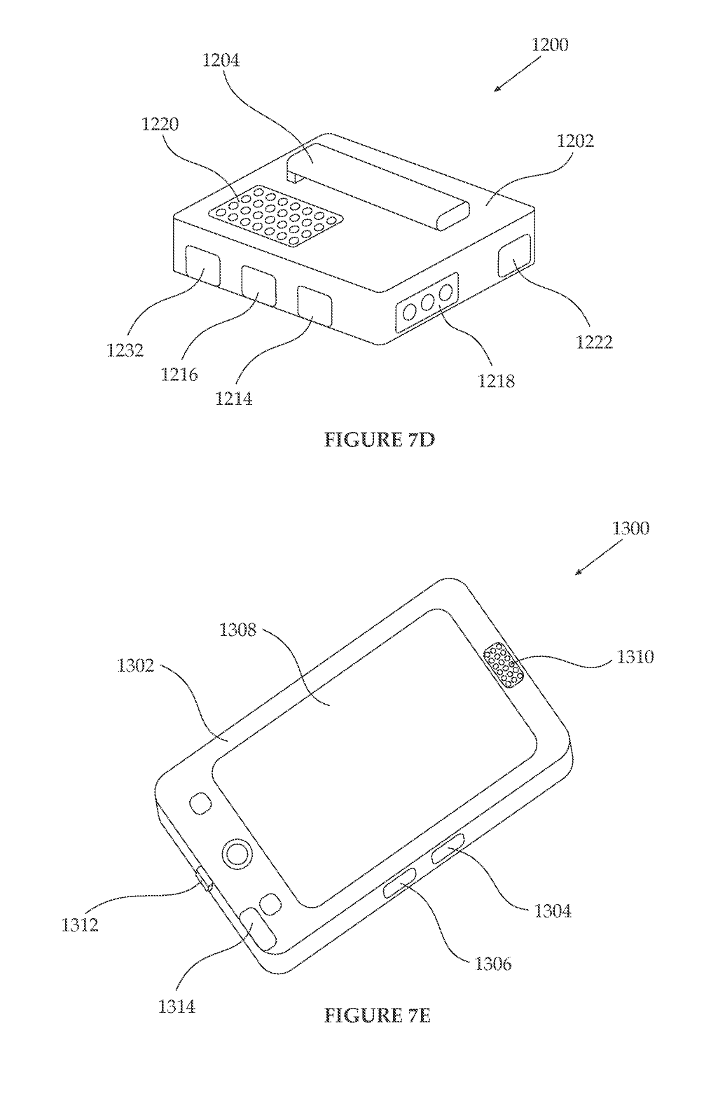

Reference is now made to FIGS. 7A-7E, which are pictorial illustrations of additional embodiments of a monitoring unit forming part of a baby monitoring device according to embodiments of the teachings herein.

As seen in FIG. 7A, a monitoring unit 900 comprises a pendant 902 configured to be worn on a necklace 904 around the user's neck. Formed on pendant 902 are a receiver 914, a processor 916, a visual indicator 918, an aural indicator 920, a tactile indicator 922, and a battery 932, all configured and operative described for equivalent parts hereinabove with reference to FIG. 2.

It is appreciated that visual indicator 918 is placed on a portion of pendant 902 visible by the wearer, and a tactile indicator 922 is formed on a portion of pendant 902 which engages the wearer's skin. It is further appreciated the external surface of pendant 902, visible by people looking at the wearer of pendant 902, may have a decorative appearance, such as that of a piece of jewelry.

Turning to FIG. 7B, it is seen that a monitoring unit 1000 comprises an intercom unit 1002 configured to be placed on the safe stable location, such as a table (not shown), for example on a desk or at the nurses' station. One part of intercom unit 1002 are a receiver 1014, a processor 1016, a visual indicator 1018, an aural indicator 1020, and a battery 1032, all configured and operative described for equivalent parts hereinabove with reference to FIG. 2.

It is appreciated that in the context of an intercom unit 1002, a tactile indicator, such a tactile indicator 222 of FIG. 2, would not be useful, since intercom unit 1002 is not to worn on a human's body and therefore vibration caused by a tactile indicator would not be felt by the baby's caretaker. That said, visual indicator 1018 and aural indicator 1020 would have the same functionality as described hereinabove with reference to FIG. 2, and would be sufficient to alert the nurse's or caretaker's attention to an emergency situation. As described hereinabove, visual indicator 1018 is placed on a portion intercom unit 1002 visible by the user.

It is further appreciated that battery 1032 of intercom unit 1002 may be replaced by an AC/DC connection for powering the components of intercom unit 1002 via an electrical socket.

Referring now to FIG. 7C, a monitoring unit 1100 comprises a pen 1102 configured to be placed in a user's pocket. Forming part of pen 1102 are a receiver 1114, a processor 1116, a visual indicator 1118, an aural indicator 1120, a tactile indicator 1122, and a battery 1132, all configured and operative described for equivalent parts hereinabove with reference to FIG. 2.

It is appreciated that visual indicator 1118 is placed on a portion of pen 1102 visible by the wearer, for example on clip 1140 used for clipping the pen onto the user's pocket, and tactile indicator 1122 is formed on a portion of pen 1102 which engages the wearer's skin, typically via a shirt, such as on a surface 1142 of pen 1102 which is opposite clip 1140.

Turning to FIG. 7D, a monitoring unit 1200 comprises a pager 1202 configured to be clipped onto a user's clothing item using a clip 1204. Forming part of pager 1202 are a receiver 1214, a processor 1216, a visual indicator 1218, an aural indicator 1220, a tactile indicator 1222, and a battery 1232, all configured and operative described for equivalent parts hereinabove with reference to FIG. 2.

It is appreciated that visual indicator 1218 is placed on a portion of pager 1202 visible by the wearer, and tactile indicator 1222 is formed on a portion of pager 1202 which engages the wearer's skin, typically via the user's clothing.

Referring now to FIG. 7E, it is seen that a mobile device 1300 includes a monitoring unit application 1302 thereby functioning as the monitoring unit of FIG. 2. A receiver 1304 of mobile device 1300 functions as receiver 214 of FIG. 2, a processor 1306 of mobile device 1300 functions as processor 216 of FIG. 2, a display 1308 of mobile device 1300 functions as visual indicator 218 of FIG. 2, a speaker 1310 of mobile device 1300 functions as an aural indicator 220 of FIG. 2, a vibrating element 1312 of mobile device 1300 functions as tactile indicator 222 of FIG. 2, and a battery 1314 of mobile device 1300 functions of battery 232 of FIG. 2.

It is appreciated that any suitable visual indication may be provided on display 1308, including a change in lighting, change in coloring, display of text, display of an image, or any other suitable visual indication.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the scope of the appended claims.

Citation or identification of any reference in this application shall not be construed as an admission that such reference is available as prior art to the invention.

Section headings are used herein to ease understanding of the specification and should not be construed as necessarily limiting.

1. A method for providing information indicative of a stress situation in a human, comprising:
positioning a pulse oximeter and an electrodermal activity (EDA) sensor to monitor a human;
at a processor functionally associated with said EDA sensor and said pulse oximeter, monitoring said human by receiving from said EDA sensor an EDA measurement signal at an EDA sensing rate;
at a processing rate, processing said received EDA measurement signals to identify an EDA indication of stress; and
if an EDA indication of stress is identified:
activating said pulse oximeter to determine a measure of at least one of pulse and blood oxygenation of the human;
at said processor, receiving from said pulse oximeter at least one pulse oximeter signal related to said determined measure of at least one of pulse and blood oxygenation;
at said processor, processing said received pulse oximeter signal to identify a pulse oximeter indication of stress; and
if a pulse oximeter indication of stress is identified, said processor providing an alarm signal.

2. The method of claim 1, wherein said providing an alarm signal comprises at least one of:
providing a visual alarm from a visual indicator functionally associated with said processor;
providing an audible alarm from an aural indicator functionally associated with said processor; and
automatically activating a wireless transmitter functionally associated with said processor to wirelessly transmit an alarm signal to a remote monitoring unit.

3. The method of claim 2, also comprising in said remote monitoring unit, providing an alarm perceivable to a human.

4. The method of claim 2, wherein said remote monitoring unit includes at least one of:
a component suitable to be worn by a person;
a component configured to be clipped onto clothes of a person; and
a component configured to be carried in a pocket of a person.

5. The method of claim 2, wherein said remote monitoring unit comprises a component not configured to be ordinarily worn by a person, the method also comprising placing said component not configured to be worn by a person in the vicinity of a person.

6. The method of claim 2, also comprising, during said monitoring of said human, if no EDA indication of stress is identified, wirelessly transmitting an all-clear signal to said monitoring unit.

7. The method of claim 2, wherein said pulse oximeter operates with power from an energy storage unit, the method also comprising:
during said monitoring of said human, monitoring a charge state of said energy storage unit; and
if said charge state of said energy storage unit is below a charge threshold, said processor automatically activating said wireless transmitter to wirelessly transmit a low battery signal to said remote monitoring unit.

8. The method of claim 2, also comprising:
in said remote monitoring unit, monitoring quality of signals received from said wireless transmitter; and
if said quality of said signals is insufficient, providing an insufficient signal indication perceivable to a human.

9. The method of claim 1, wherein said measure of blood oxygenation comprises a blood oxygenation percentage.

10. The method of claim 1, wherein said pulse oximeter indication of stress comprises at least one of:
a blood oxygenation measure of said pulse oximeter signal being below a blood oxygenation threshold;
a pulse rate measure of said pulse oximeter signal being below a low pulse rate threshold; and
a pulse rate measure of said pulse oximeter signal being above a high pulse rate threshold.

11. The method claim 1, also comprising indicating whether said pulse oximeter indication is indicative of stress stemming from a problem in blood oxygenation of said human or of stress stemming from a problem in pulse rate of said human.

12. The method of claim 1, also comprising storing said EDA measurement signals and/or said measures of pulse and/or blood oxygenation in a memory.

13. The method of claim 1, also comprising, during said monitoring of said human, if no EDA indication of stress is identified, keeping said pulse oximeter in an inactive energy-saving state.

14. A device useful for providing information indicative of a stress situation in a human human, comprising:
a pulse-oximeter configured for determining a measure of at least one of pulse and blood oxygenation of a human and producing a pulse oximeter signal related to a said determined measure of at least one of pulse and blood oxygenation;
an electrodermal activity (EDA) sensor configured for determining electrodermal activity of a human at an EDA sensing rate and producing an EDA measurement signal related to said determined electrodermal activity;
a processor functionally associated with said pulse oximeter and with said EDA sensor; and
a wireless transmitter functionally associated with said processor,
wherein said processor is configured to:
receive from said EDA sensor an EDA measurement signal at said EDA sensing rate, at a processing rate, process said received EDA measurement signals to identify an EDA indication of stress,
if an EDA indication of stress is identified, activate said pulse oximeter to determine a measure of at least one of pulse and blood oxygenation of the human,
receive from said pulse oximeter at least one pulse oximeter signal related to said determined measure of at least one of pulse and blood oxygenation,
process said received pulse oximeter signal to identify a pulse oximeter indication of stress, and
if a pulse oximeter indication of stress is identified, automatically provide an alarm signal.

15. The device of claim 14, also comprising:
an indicator configured, upon provision of said alarm signal, to provide an indication perceivable to a human.

16. The device of claim 14, also comprising a remote monitoring unit, including:
a wireless receiver configured to receive transmissions from said wireless transmitter; and
an alarm indicator, configured to provide an alarm perceivable to a human upon receipt of a said alarm signal by said wireless receiver.

17. The device of claim 16, wherein said remote monitoring unit comprises at least one of:
a component suitable to be worn by a person;
a component configured to be clipped onto clothes of a person;
a component configured to be carried the pocket of a person; and
a component not configured to be ordinarily worn by a person.

18-22. (canceled)

23. The device of claim 14, wherein said alarm signal indicates whether said pulse oximeter indication is indicative of stress stemming from a problem in blood oxygenation of said human or of stress stemming from a problem in pulse rate of said human.

24-27. (canceled)

28. The device of claim 14, configured to keep said pulse oximeter in an inactive energy-saving state as long as no EDA indication of stress is identified.

29. A method for providing information indicative of a reason for a stress situation in a human, comprising:
positioning an electrodermal activity (EDA) sensor to monitor a human;
at a processor functionally associated with said EDA sensor, receiving from said EDA sensor an EDA measurement signal at a EDA sensing rate;
at a processing rate, processing said received EDA measurement signals to identify an EDA level category;
based on said identified EDA level category providing an appropriate signal, wherein:
if said received EDA measurement is identified to be in a first EDA level category, providing an alarm indicating medical stress,
if said received EDA measurement is identified to be in a second EDA level category, providing a signal indicating non-medical stress, and
if said received EDA measurement is identified to be in a third EDA level category, providing a signal indicating that no stress is identified.

30. (canceled)
